(19) United States
(12) Patent Application Publication
(10) Pub. No.: US 2024/0084295 A1
(43) Pub. Date: Mar. 14, 2024

(54) Novel Synthesis of Phosphorodithioate Oligonucleotides

(71) Applicant: Hoffmann-La Roche Inc., Little Falls, NJ (US)

(72) Inventors: Martin Bollmark, Basel (CH); Walter Fernando Sehgelmeble Torrejon, Basel (CH); Filippo Sladojevich, Basel (CH); Ulf Tedebark, Basel (CH)

(21) Appl. No.: 18/331,536

(22) Filed: Aug. 29, 2023

Related U.S. Application Data
(63) Continuation of application No. PCT/EP2021/084316, filed on Dec. 6, 2021.

(30) Foreign Application Priority Data
Dec. 8, 2020 (EP) 20212441.8

(51) Int. Cl. C12N 15/11; C07H 19/10; C07H 19/20

(52) U.S. Cl. CPC C12N 15/111; C07H 19/10; C07H 19/20; C12N 2310/313; C12N 2310/3231; C12N 2330/30

(57) ABSTRACT

The invention provides the use of a compound of formula (I), as defined, for the preparation of an oligonucleotide comprising at least one phosphorodithioate internucleoside linkage. Various synthesis methods using compounds of formula (I) are provided.

### CROSS REFERENCE TO RELATED APPLICATIONS

This application is a Bypass continuation patent application, filed under 35 USC § 111 claiming priority to International Patent Application No. PCT/EP2021/084316 filed on Dec. 6, 2021, which claims benefit of and priority to European Patent Application No. 20212441.8 filed on Dec. 8, 2020, which are incorporated herein by reference in their entirety.

### REFERENCE TO SEQUENCE LISTING

The instant application contains a Sequence Listing which has been submitted in XML format via EFS-Web and is hereby incorporated by reference in its entirety. Said XML copy, created on Jun. 8, 2023, is named 067211.021US1.xml and is 46 kilobytes in size.

### BACKGROUND

The use of synthetic oligonucleotides as therapeutic agents has witnessed remarkable progress over recent decades leading to the development of molecules acting by diverse mechanisms including RNase H activating gapmers, splice switching oligonucleotides, microRNA inhibitors, siRNA or aptamers (S. T. Crooke, Antisense drug technology: principles, strategies, and applications, 2nd ed. ed., Boca Raton, Fla.: CRC Press, 2008). However, oligonucleotides are inherently unstable towards nucleolytic degradation in biological systems. Furthermore, they show a highly unfavorable pharmacokinetic behavior.

In order to improve on these drawbacks a wide variety of chemical modifications have been investigated in recent decades. Arguably one of the most successful modification is the introduction of phosphorothioate linkages, where one of the non-bridging phosphate oxygen atoms is replaced with a sulfur atom (F. Eckstein, Antisense and Nucleic Acid Drug Development 2009, 10, 117-121.). Such phosphorothioate oligodeoxynucleotides show an increased protein binding as well as a distinctly higher stability to nucleolytic degradation and thus a substantially higher half-life in plasma, tissues and cells than their unmodified phosphodiester analogues. These crucial features have allowed for the development of the first generation of oligonucleotide therapeutics as well as opened the door for their further improvement through later generation modifications such as Locked Nucleic Acids (LNAs).

Replacement of a phosphodiester linkage with a phosphorothioate, however, creates a chiral center at the phosphorous atom. As a consequence, all approved phosphorothioate oligonucleotide therapeutics are used as mixtures of a huge amount of diastereoisomeric compounds, which all potentially have different (and possibly opposing) physiochemical and pharmacological properties.

A few oligomer syntheses of phosphorodithioates using H-phosphonithioates have been reported, for instance Kamaike et al. Tetrahedron 2006, 62, 11814; Kamaike et al. Tetrahedron Lett. 2004, 45, 5803; Kamaike et al. Nucleic Acids Symp Ser. 2003, 3, 93; Seeberger et al. Tetrahedron 1999, 55, 5759; Zain et al. Nucleosides, Nucleotides and Nucleic Acids, 1997, 16, 1661 and Greef et al. Tetrahedron Lett. 1996, 37, 4451.

It has been surprisingly found by the inventors that improved preparation of an oligonucleotide comprising at least one phosphorodithioate internucleoside linkage can be achieved using H-phosphothionates (comprising a P-H moiety at the phosphorothioate linkage). The preparation is particularly well-suited to synthesis on a solid support and can be readily combined with standard oligonucleotide syntheses (e.g. from cyanoethyl protected amidites). Additionally, the formation of impurities—in particular phosphorothioate impurities—can be reduced.

### STATEMENT OF THE INVENTION

The invention relates to the use of a compound of formula (I):

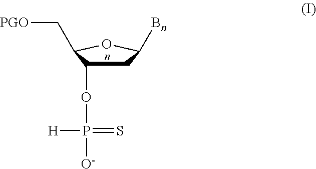

for the preparation of an oligonucleotide comprising at least one phosphorodithioate internucleoside linkage, wherein PG is a hydroxyl-protecting group, Bn is a nucleobase that can be natural or non-natural, and wherein ribose n is modified at the 2′ position of its ring.

The invention also relates to a method for the preparation of an oligonucleotide containing at least one phosphorodithioate internucleoside linkage, said method comprising the steps of:
a) coupling a compound of formula (I):

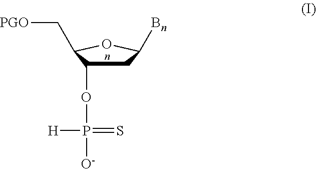

wherein—in formula (I)—PG is a hydroxyl-protecting group, Bn is a nucleobase that can be natural or non-natural, and wherein the ribose n is modified at the 2′ position of its ring, with a compound of formula (II):

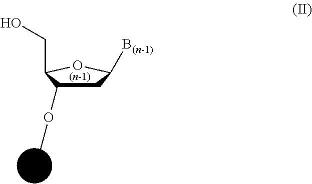

wherein—in formula (II)—B(n−1) is a nucleobase that can be natural or non-natural, and wherein ribose (n−1) can be unmodified or modified at the 2′ position of its ring,
in the presence of a coupling agent, to obtain a compound of formula (III):

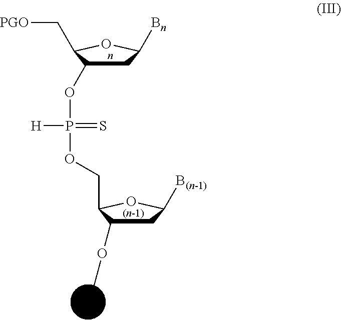

The invention also relates to a phosphonothioate monoester selected from the group consisting of:

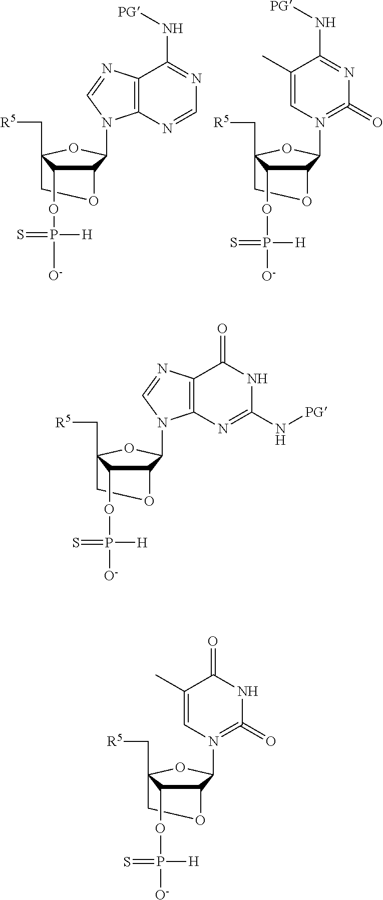

wherein PG′ is an amino-protecting group and R⁵ is H or a hydroxyl-protecting group.

The invention further relates to a phosphonothioate monoester salt selected from the group consisting of:

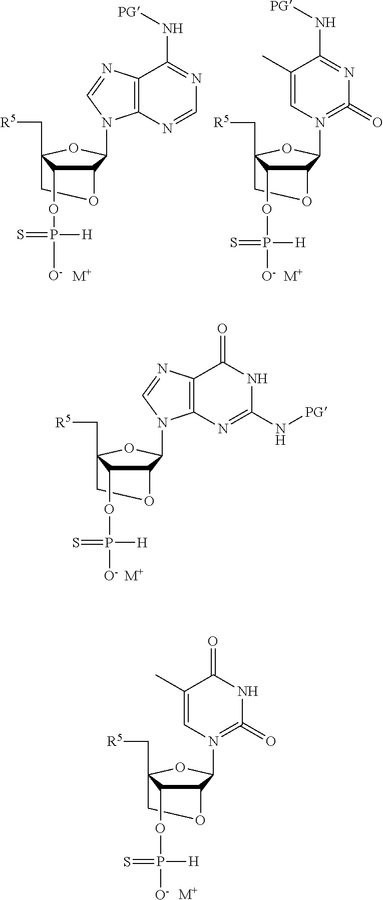

wherein PG′ is an amino-protecting group, M⁺ is a monovalent cation and R⁵ is H or a hydroxyl-protecting group.

### FIGURES

FIG. 1 shows how H-phosphonothioates may be used in a coupling sequence.

FIG. 2 shows oligomer synthesis using compounds of formula (I) with sulfurization (thioalkylation) after each coupling

FIG. 3 shows oligomer synthesis using compounds of formula (I) with final sulfurization

FIG. 4 shows a particular example of coupling of a H-phosphonothioate on a solid support.

### DEFINITIONS

In the present description the term “alkyl”, alone or in combination, signifies a straight-chain or branched-chain alkyl group with 1 to 8 carbon atoms, particularly a straight or branched-chain alkyl group with 1 to 6 carbon atoms and more particularly a straight or branched-chain alkyl group with 1 to 4 carbon atoms. Examples of straight-chain and branched-chain C₁-C₈ alkyl groups are methyl, ethyl, propyl, isopropyl, butyl, isobutyl, tert.-butyl, the isomeric pentyls, the isomeric hexyls, the isomeric heptyls and the isomeric octyls, particularly methyl, ethyl, propyl, butyl and pentyl. Particular examples of alkyl are methyl, ethyl and propyl.

The term “cycloalkyl”, alone or in combination, signifies a cycloalkyl ring with 3 to 8 carbon atoms and particularly a cycloalkyl ring with 3 to 6 carbon atoms. Examples of cycloalkyl are cyclopropyl, cyclobutyl, cyclopentyl, cyclohexyl, cycloheptyl and cyclooctyl, more particularly cyclopropyl and cyclobutyl. A particular example of “cycloalkyl” is cyclopropyl.

The term “alkoxy”, alone or in combination, signifies a group of the formula alkyl-O- in which the term “alkyl” has the previously given significance, such as methoxy, ethoxy, n-propoxy, isopropoxy, n-butoxy, isobutoxy, sec.butoxy and tert.butoxy. Particular “alkoxy” are methoxy and ethoxy. Methoxyethoxy is a particular example of “alkoxyalkoxy”.

The term “oxy”, alone or in combination, signifies the —O-group.

The term “alkenyl”, alone or in combination, signifies a straight-chain or branched hydrocarbon residue comprising an olefinic bond and up to 8, preferably up to 6, particularly preferred up to 4 carbon atoms. Examples of alkenyl groups are ethenyl, 1-propenyl, 2-propenyl, isopropenyl, 1-butenyl, 2-butenyl, 3-butenyl and isobutenyl.

The term “alkynyl”, alone or in combination, signifies a straight-chain or branched hydrocarbon residue comprising a triple bond and up to 8, particularly 2 carbon atoms.

The terms “halogen” or “halo”, alone or in combination, signifies fluorine, chlorine, bromine or iodine and particularly fluorine, chlorine or bromine, more particularly fluorine. The term “halo”, in combination with another group, denotes the substitution of said group with at least one halogen, particularly substituted with one to five halogens, particularly one to four halogens, i.e. one, two, three or four halogens.

The term “haloalkyl”, alone or in combination, denotes an alkyl group substituted with at least one halogen, particularly substituted with one to five halogens, particularly one to three halogens. Examples of haloalkyl include monofluoro-, difluoro- or trifluoro-methyl, -ethyl or -propyl, for example 3,3,3-trifluoropropyl, 2-fluoroethyl, 2,2,2-trifluoroethyl, fluoromethyl or trifluoromethyl. Fluoromethyl, difluoromethyl and trifluoromethyl are particular “haloalkyl”.

The term “halocycloalkyl”, alone or in combination, denotes a cycloalkyl group as defined above substituted with at least one halogen, particularly substituted with one to five halogens, particularly one to three halogens. Particular example of “halocycloalkyl” are halocyclopropyl, in particular fluorocyclopropyl, difluorocyclopropyl and trifluorocyclopropyl.

The terms “hydroxyl” and “hydroxy”, alone or in combination, signify the —OH group.

The terms “thiohydroxyl” and “thiohydroxy”, alone or in combination, signify the —SH group.

The term “carbonyl”, alone or in combination, signifies the —C(O)-group.

The term “carboxy” or “carboxyl”, alone or in combination, signifies the —COOH group.

The term “amino”, alone or in combination, signifies the primary amino group (—NH₂), the secondary amino group (—NH—), or the tertiary amino group (—N—).

The term “alkylamino”, alone or in combination, signifies an amino group as defined above substituted with one or two alkyl groups as defined above.

The term “sulfonyl”, alone or in combination, means the —SO₂ group.

The term “sulfinyl”, alone or in combination, signifies the —SO—group.

The term “sulfanyl”, alone or in combination, signifies the —S—group.

The term “cyano”, alone or in combination, signifies the —CN group.

The term “azido”, alone or in combination, signifies the —N₃ group.

The term “nitro”, alone or in combination, signifies the NO₂ group.

The term “formyl”, alone or in combination, signifies the —C(O)H group.

The term “carbamoyl”, alone or in combination, signifies the —C(O)NH₂ group.

The term “cabamido”, alone or in combination, signifies the —NH—C(O)—NH₂ group.

The term “aryl”, alone or in combination, denotes a monovalent aromatic carbocyclic mono- or bicyclic ring system comprising 6 to 10 carbon ring atoms, optionally substituted with 1 to 3 substituents independently selected from halogen, hydroxyl, alkyl, alkenyl, alkynyl, alkoxy, alkoxyalkyl, alkenyloxy, carboxyl, alkoxycarbonyl, alkylcarbonyl and formyl. Examples of aryl include phenyl and naphthyl, in particular phenyl.

The term “heteroaryl”, alone or in combination, denotes a monovalent aromatic heterocyclic mono- or bicyclic ring system of 5 to 12 ring atoms, comprising 1, 2, 3 or 4 heteroatoms selected from N, O and S, the remaining ring atoms being carbon, optionally substituted with 1 to 3 substituents independently selected from halogen, hydroxyl, alkyl, alkenyl, alkynyl, alkoxy, alkoxyalkyl, alkenyloxy, carboxyl, alkoxycarbonyl, alkylcarbonyl and formyl. Examples of heteroaryl include pyrrolyl, furanyl, thienyl, imidazolyl, oxazolyl, thiazolyl, triazolyl, oxadiazolyl, thiadiazolyl, tetrazolyl, pyridinyl, pyrazinyl, pyrazolyl, pyridazinyl, pyrimidinyl, triazinyl, azepinyl, diazepinyl, isoxazolyl, benzofuranyl, isothiazolyl, benzothienyl, indolyl, isoindolyl, isobenzofuranyl, benzimidazolyl, benzoxazolyl, benzoisoxazolyl, benzothiazolyl, benzoisothiazolyl, benzooxadiazolyl, benzothiadiazolyl, benzotriazolyl, purinyl, quinolinyl, isoquinolinyl, quinazolinyl, quinoxalinyl, carbazolyl or acridinyl.

The term “heterocyclyl”, alone or in combination, signifies a monovalent saturated or partly unsaturated mono- or bicyclic ring system of 4 to 12, in particular 4 to 9 ring atoms, comprising 1, 2, 3 or 4 ring heteroatoms selected from N, O and S, the remaining ring atoms being carbon, optionally substituted with 1 to 3 substituents independently selected from halogen, hydroxyl, alkyl, alkenyl, alkynyl, alkoxy, alkoxyalkyl, alkenyloxy, carboxyl, alkoxycarbonyl, alkylcarbonyl and formyl. Examples for monocyclic saturated heterocyclyl are azetidinyl, pyrrolidinyl, tetrahydrofuranyl, tetrahydro-thienyl, pyrazolidinyl, imidazolidinyl, oxazolidinyl, isoxazolidinyl, thiazolidinyl, piperidinyl, tetrahydropyranyl, tetrahydrothiopyranyl, piperazinyl, morpholinyl, thiomorpholinyl, 1,1-dioxo-thiomorpholin-4-yl, azepanyl, diazepanyl, homopiperazinyl, or oxazepanyl. Examples for bicyclic saturated heterocycloalkyl are 8-aza-bicyclo[3.2.1]octyl, quinuclidinyl, 8-oxa-3-aza-bicyclo[3.2.1]octyl, 9-aza-bicyclo[3.3.1]nonyl, 3-oxa-9-aza-bicyclo[3.3.1]nonyl, or 3-thia-9-aza-bicyclo[3.3.1]nonyl. Examples for partly unsaturated heterocycloalkyl are dihydrofuryl, imidazolinyl, dihydro-oxazolyl, tetrahydro-pyridinyl or dihydropyranyl.

The term “pharmaceutically acceptable salts” refers to those salts which retain the biological effectiveness and properties of the free bases or free acids, which are not biologically or otherwise undesirable. The salts are formed with inorganic acids such as hydrochloric acid, hydrobromic acid, sulfuric acid, nitric acid, phosphoric acid, particularly hydrochloric acid, and organic acids such as acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, N-acetylcystein. In addition, these salts may be prepared form addition of an inorganic base or an organic base to the free acid. Salts derived from an inorganic base include, but are not limited to, the sodium, potassium, lithium, ammonium, calcium, magnesium salts. Salts derived from organic bases include, but are not limited to salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, lysine, arginine, N-ethylpiperidine, piperidine, polyamine resins. The oligonucleotide of the invention can also be present in the form of zwitterions. Particularly preferred pharmaceutically acceptable salts of the invention are the sodium, lithium, potassium and trialkylammonium salts.

The term “protecting group”, alone or in combination, signifies a group which selectively blocks a reactive site in a multifunctional compound such that a chemical reaction can be carried out selectively at another unprotected reactive site. Protecting groups can be removed. Exemplary protecting groups are amino-protecting groups, carboxy-protecting groups or hydroxyl-protecting groups.

“Phosphate protecting group” is a protecting group of the phosphate group. Examples of phosphate protecting group are 2-cyanoethyl and methyl. A particular example of phosphate protecting group is 2-cyanoethyl.

“Hydroxyl protecting group” is a protecting group of the hydroxyl group and is also used to protect thiol groups. Examples of hydroxyl protecting groups are acetyl (Ac), benzoyl (Bz), benzyl (Bn), β-methoxyethoxymethyl ether (MEM), dimethoxytrityl (or bis-(4-methoxyphenyl)phenylmethyl) (DMT), trimethoxytrityl (or tris-(4-methoxyphenyl)phenylmethyl) (TMT), methoxymethyl ether (MOM), methoxytrityl [(4-methoxyphenyl)diphenylmethyl (MMT), p-methoxybenzyl ether (PMB), methylthiomethyl ether, pivaloyl (Piv), tetrahydropyranyl (THP), tetrahydrofuran (THF), trityl or triphenylmethyl (Tr), silyl ether (for example trimethylsilyl (TMS), tert-butyldimethylsilyl (TBDMS), tri-iso-propylsilyloxymethyl (TOM) and triisopropylsilyl (TIPS) ethers), methyl ethers and ethoxyethyl ethers (EE). Particular examples of hydroxyl protecting group are DMT and TMT, in particular DMT.

“Thiohydroxyl protecting group” is a protecting group of the thiohydroxyl group. Examples of thiohydroxyl protecting groups are those of the “hydroxyl protecting group”.

If one of the starting materials or compounds of the invention contain one or more functional groups which are not stable or are reactive under the reaction conditions of one or more reaction steps, appropriate protecting groups (as described e.g. in “Protective Groups in Organic Chemistry” by T. W. Greene and P. G. M. Wuts, 3^{rd} Ed., 1999, Wiley, New York) can be introduced before the critical step applying methods well known in the art. Such protecting groups can be removed at a later stage of the synthesis using standard methods described in the literature. Examples of protecting groups are tert-butoxycarbonyl (Boc), 9-fluorenylmethyl carbamate (Fmoc), 2-trimethylsilylethyl carbamate (Teoc), carbobenzyloxy (Cbz) and p-methoxybenzyloxycarbonyl (Moz).

Standard amino-protecting groups which may be used for the heterocyclic bases include benzoyl, acetyl, dimethylformamidyl, isobutiryl, phenoxyacetyl, and isopropylphenoxyacetyl.

The compounds described herein can contain several asymmetric centers and can be present in the form of optically pure enantiomers, mixtures of enantiomers such as, for example, racemates, mixtures of diastereoisomers, diastereoisomeric racemates or mixtures of diastereoisomeric racemates.

### Oligonucleotide

The term “oligonucleotide” as used herein is defined as it is generally understood by the skilled person as a molecule comprising two or more covalently linked nucleosides. Such covalently bound nucleosides may also be referred to as nucleic acid molecules or oligomers. Oligonucleotides are commonly made in the laboratory by solid-phase chemical synthesis followed by purification. When referring to a sequence of the oligonucleotide, reference is made to the sequence or order of nucleobase moieties, or modifications thereof, of the covalently linked nucleotides or nucleosides. The oligonucleotide of the invention is man-made, and is chemically synthesized, and is typically purified or isolated. The oligonucleotide of the invention may comprise one or more modified nucleosides or nucleotides.

### Antisense Oligonucleotides

The term “Antisense oligonucleotide” as used herein is defined as oligonucleotides capable of modulating expression of a target gene by hybridizing to a target nucleic acid, in particular to a contiguous sequence on a target nucleic acid. The antisense oligonucleotides are not essentially double stranded and are therefore not siRNAs or shRNAs. Preferably, the antisense oligonucleotides of the present invention are single stranded. It is understood that single stranded oligonucleotides of the present invention can form hairpins or intermolecular duplex structures (duplex between two molecules of the same oligonucleotide), as long as the degree of intra or inter self-complementarity is less than 50% across of the full length of the oligonucleotide.

### Contiguous Nucleotide Sequence

The term “contiguous nucleotide sequence” refers to the region of the oligonucleotide which is complementary to, such as fully complementary to, the target nucleic acid. The term is used interchangeably herein with the term “contiguous nucleobase sequence” and the term “oligonucleotide motif sequence”. In some embodiments all the nucleotides of the oligonucleotide constitute the contiguous nucleotide sequence. In some embodiments the oligonucleotide comprises the contiguous nucleotide sequence, such as a F-G-F′ gapmer region, and may optionally comprise further nucleotide(s), for example a nucleotide linker region which may be used to attach a functional group to the contiguous nucleotide sequence, e.g. region D or D′. The nucleotide linker region may or may not be complementary to the target nucleic acid. The antisense oligonucleotide mixmer referred to herein may comprise or may consist of the contiguous nucleotide sequence.

### Nucleotides

Nucleotides are the building blocks of oligonucleotides and polynucleotides, and for the purposes of the present invention include both naturally occurring and non-naturally occurring nucleotides. In nature, nucleotides, such as DNA and RNA nucleotides comprise a ribose sugar moiety, a nucleobase moiety and one or more phosphate groups (which is absent in nucleosides). Nucleosides and nucleotides may also interchangeably be referred to as “units” or “monomers”.

### Modified Nucleoside

The term “modified nucleoside” or “nucleoside modification” as used herein refers to nucleosides modified as compared to the equivalent DNA or RNA nucleoside by the introduction of one or more modifications of the sugar moiety or the (nucleo)base moiety. In a preferred embodiment the modified nucleoside comprises a modified sugar moiety. The term modified nucleoside may also be used herein interchangeably with the term “nucleoside analogue” or modified “units” or modified “monomers”. Nucleosides with an unmodified DNA or RNA sugar moiety are termed DNA or RNA nucleosides herein. Nucleosides with modifications in the base region of the DNA or RNA nucleoside are still generally termed DNA or RNA if they allow Watson Crick base pairing.

### Modified Internucleoside Linkage

The term “modified internucleoside linkage” is defined as generally understood by the skilled person as linkages other than phosphodiester (PO) linkages, that covalently couples two nucleosides together. The oligonucleotides of the invention may therefore comprise modified internucleoside linkages. In some embodiments, the modified internucleoside linkage increases the nuclease resistance of the oligonucleotide compared to a phosphodiester linkage. For naturally occurring oligonucleotides, the internucleoside linkage includes phosphate groups creating a phosphodiester bond between adjacent nucleosides. Modified internucleoside linkages are particularly useful in stabilizing oligonucleotides for in vivo use, and may serve to protect against nuclease cleavage at regions of DNA or RNA nucleosides in the oligonucleotide of the invention, for example within the gap region of a gapmer oligonucleotide, as well as in regions of modified nucleosides, such as region F and F′.

In an embodiment, the oligonucleotide comprises one or more internucleoside linkages modified from the natural phosphodiester, such one or more modified internucleoside linkages that is for example more resistant to nuclease attack. Nuclease resistance may be determined by incubating the oligonucleotide in blood serum or by using a nuclease resistance assay (e.g. snake venom phosphodiesterase (SVPD)), both are well known in the art. Internucleoside linkages which are capable of enhancing the nuclease resistance of an oligonucleotide are referred to as nuclease resistant internucleoside linkages. In some embodiments at least 50% of the internucleoside linkages in the oligonucleotide, or contiguous nucleotide sequence thereof, are modified, such as at least 60%, such as at least 70%, such as at least 80 or such as at least 90% of the internucleoside linkages in the oligonucleotide, or contiguous nucleotide sequence thereof, are nuclease resistant internucleoside linkages. In some embodiments all of the internucleoside linkages of the oligonucleotide, or contiguous nucleotide sequence thereof, are nuclease resistant internucleoside linkages. It will be recognized that, in some embodiments the nucleosides which link the oligonucleotide of the invention to a non-nucleotide functional group, such as a conjugate, may be phosphodiester.

The compounds of the present invention are used for the preparation of an oligonucleotide comprising at least one phosphorodithioate internucleoside linkage. Phosphorodithioate linkages are internucleoside phosphate linkages where both of the non-bridging oxygen atoms have been substituted with a sulfur atom.

Phosphorodithioate internucleoside linkages are particularly useful due to nuclease resistance, beneficial pharmacokinetics and ease of manufacture. In some embodiments at least 50% of the internucleoside linkages in the oligonucleotide, or contiguous nucleotide sequence thereof, are phosphorodithioate, such as at least 60%, such as at least 70%, such as at least 80% or such as at least 90% of the internucleoside linkages in the oligonucleotide, or contiguous nucleotide sequence thereof, are phosphorodithioate. In some embodiments, other than the phosphorodithioate internucleoside linkages, all of the internucleoside linkages of the oligonucleotide, or contiguous nucleotide sequence thereof, are phosphorothioate. In some embodiments, the oligonucleotide of the invention comprises both phosphorodithioate internucleoside linkages and at least one phosphodiester linkage, such as 2, 3 or 4 phosphodiester linkages, in addition to the phosphorodithioate linkage(s). In a gapmer oligonucleotide, phosphodiester linkages, when present, are suitably not located between contiguous DNA nucleosides in the gap region G.

Nuclease resistant linkages, such as phosphorodithioate linkages, are particularly useful in oligonucleotide regions capable of recruiting nuclease when forming a duplex with the target nucleic acid, such as region G for gapmers. Phosphorodithioate linkages may, however, also be useful in non-nuclease recruiting regions and/or affinity enhancing regions such as regions F and F′ for gapmers. Gapmer oligonucleotides may, in some embodiments comprise one or more phosphodiester linkages in region F or F′, or both region F and F′, which the internucleoside linkage in region G may be fully phosphorodithioate.

Advantageously, all the internucleoside linkages in the contiguous nucleotide sequence of the oligonucleotide, or all the internucleoside linkages of the oligonucleotide, are phosphorodithioate linkages. Alternatively, the oligonucleotide prepared according to the invention comprises at least one phosphorodithioate and at least one phosphorothioate internucleoside linkage. As a further alternative, the oligonucleotide prepared according to the invention comprises at least one phosphorodithioate, at least one phosphorothioate and/or at least one phosphodiester internucleoside linkage.

It is recognized that, as disclosed in EP 2 742 135, oligonucleotides may comprise other internucleoside linkages (other than phosphodiester, phosphorothioate or phosphorodithioate).

### Stereodefined Internucleoside Linkages

A stereodefined internucleoside linkage is a chiral internucleoside linkage having a diastereoisomeric excess for one of its two diastereomeric forms, Rp or Sp.

It should be recognized that stereoselective oligonucleotide synthesis methods used in the art typically provide at least about 90% or at least about 95% diastereoselectivity at each chiral internucleoside linkage, and as such up to about 10%, such as about 5% of oligonucleotide molecules may have the alternative diastereoisomeric form.

In some embodiments the diastereoisomeric ratio of each stereodefined chiral internucleoside linkage is at least about 90:10. In some embodiments the diastereoisomeric ratio of each chiral internucleoside linkage is at least about 95:5.

The stereodefined phosphorothioate linkage is a particular example of stereodefined internucleoside linkage.

### Stereodefined Phosphorothioate Linkage

A stereodefined phosphorothioate linkage is a phosphorothioate linkage having a diastereomeric excess for one of its two diastereosiomeric forms, Rp or Sp.

The Rp and Sp configurations of the phosphorothioate internucleoside linkages are presented below

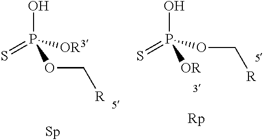

Where the 3′ R group represents the 3′ position of the adjacent nucleoside (a 5′ nucleoside), and the 5′ R group represents the 5′ position of the adjacent nucleoside (a 3′ nucleoside).

Rp internucleoside linkages may also be represented as srP, and Sp internucleoside linkages may be represented as ssP herein.

In a particular embodiment, the diastereomeric ratio of each stereodefined phosphorothioate linkage is at least about 90:10 or at least 95:5.

In some embodiments the diastereomeric ratio of each stereodefined phosphorothioate linkage is at least about 97:3. In some embodiments the diastereomeric ratio of each stereodefined phosphorothioate linkage is at least about 98:2. In some embodiments the diastereomeric ratio of each stereodefined phosphorothioate linkage is at least about 99:1.

In some embodiments a stereodefined internucleoside linkage is in the same diastereomeric form (Rp or Sp) in at least 97%, such as at least 98%, such as at least 99%, or (essentially) all of the oligonucleotide molecules present in a population of the oligonucleotide molecule.

Diastereomeric purity can be measured in a model system only having an achiral backbone (i.e. phosphodiesters). It is possible to measure the diastereomeric purity of each monomer by e.g. coupling a monomer having a stereodefine internucleoside linkage to the following model-system “5′ t-po-t-po-t-po 3”. The result of this will then give: 5′ DMTr-t-srp-t-po-t-po-t-po 3′ or 5′ DMTr-t-ssp-t-po-t-po-t-po 3′ which can be separated using HPLC. The diastereomeric purity is determined by integrating the UV signal from the two possible diastereoisomers and giving a ratio of these e.g. 98:2, 99:1 or >99:1.

It will be understood that the diastereomeric purity of a specific single diastereoisomer (a single stereodefined oligonucleotide molecule) will be a function of the coupling selectivity for the defined stereocenter at each internucleoside position, and the number of stereodefined internucleoside linkages to be introduced. By way of example, if the coupling selectivity at each position is 97%, the resulting purity of the stereodefined oligonucleotide with 15 stereodefined internucleoside linkages will be 0.97 15 , i.e. 63% of the desired diastereoisomer as compared to 37% of the other diastereoisomers. The purity of the defined diastereoisomer may after synthesis be improved by purification, for example by HPLC, such as ion exchange chromatography or reverse phase chromatography.

In some embodiments, a stereodefined oligonucleotide refers to a population of an oligonucleotide wherein at least about 40%, such as at least about 50% of the population is of the desired diastereoisomer.

Alternatively stated, in some embodiments, a stereodefined oligonucleotide refers to a population of oligonucleotides wherein at least about 40%, such as at least about 50%, of the population consists of the desired (specific) stereodefined internucleoside linkage motifs (also termed stereodefined motif).

For stereodefined oligonucleotides which comprise both stereorandom and stereodefined internucleoside chiral centers, the purity of the stereodefined oligonucleotide is determined with reference to the % of the population of the oligonucleotide which retains the desired stereodefined internucleoside linkage motif(s), the stereorandom linkages being disregarded in the calculation.

### Modified Oligonucleotide

The term modified oligonucleotide describes an oligonucleotide comprising one or more sugar-modified nucleosides and/or modified internucleoside linkages. The term chimeric” oligonucleotide is a term that has been used in the literature to describe oligonucleotides with modified nucleosides.

### Stereodefined Oligonucleotide

A stereodefined oligonucleotide is an oligonucleotide wherein at least one of the internucleoside linkages is a stereodefined internucleoside linkage.

A stereodefined phosphorothioate oligonucleotide is an oligonucleotide wherein at least one of the internucleoside linkages is a stereodefined phosphorothioate internucleoside linkage.

### Complementarity

The term “complementarity” describes the capacity for Watson-Crick base-pairing of nucleosides/nucleotides. Watson-Crick base pairs are guanine (G)-cytosine (C) and adenine (A)—thymine (T)/uracil (U). It will be understood that oligonucleotides may comprise nucleosides with modified nucleobases, for example 5-methyl cytosine is often used in place of cytosine, and as such the term complementarity encompasses Watson Crick base-paring between non-modified and modified nucleobases (see for example Hirao et al (2012) Accounts of Chemical Research vol 45 page 2055 and Bergstrom (2009) Current Protocols in Nucleic Acid Chemistry Suppl. 37 1.4.1).

The term “% complementary” as used herein, refers to the proportion of nucleotides in a contiguous nucleotide sequence in a nucleic acid molecule (e.g. oligonucleotide) which, at a given position, are complementary to (i.e. form Watson Crick base pairs with) a contiguous nucleotide sequence, at a given position of a separate nucleic acid molecule (e.g. the target nucleic acid). The percentage is calculated by counting the number of aligned bases that form pairs between the two sequences (when aligned with the target sequence 5′-3′ and the oligonucleotide sequence from 3′-5′), dividing by the total number of nucleotides in the oligonucleotide and multiplying by 100. In such a comparison a nucleobase/nucleotide which does not align (form a base pair) is termed a mismatch. Preferably, insertions and deletions are not allowed in the calculation of % complementarity of a contiguous nucleotide sequence.

The term “fully complementary”, refers to 100% complementarity.

### Hybridization

The term “hybridizing” or “hybridizes” as used herein is to be understood as two nucleic acid strands (e.g. an oligonucleotide and a target nucleic acid) forming hydrogen bonds between base pairs on opposite strands thereby forming a duplex. The affinity of the binding between two nucleic acid strands is the strength of the hybridization. It is often described in terms of the melting temperature (Tₘ) defined as the temperature at which half of the oligonucleotides are duplexed with the target nucleic acid. At physiological conditions Tₘ is not strictly proportional to the affinity (Mergny and Lacroix, 2003, Oligonucleotides 13:515-537). The standard state Gibbs free energy ΔG° is a more accurate representation of binding affinity and is related to the dissociation constant (K_{d}) of the reaction by ΔG°=−RTIn(K_{d}), where R is the gas constant and T is the absolute temperature. Therefore, a very low ΔG° of the reaction between an oligonucleotide and the target nucleic acid reflects a strong hybridization between the oligonucleotide and target nucleic acid. ΔG° is the energy associated with a reaction where aqueous concentrations are 1M, the pH is 7, and the temperature is 37° C. The hybridization of oligonucleotides to a target nucleic acid is a spontaneous reaction and for spontaneous reactions ΔG° is less than zero. ΔG° can be measured experimentally, for example, by use of the isothermal titration calorimetry (ITC) method as described in Hansen et al., 1965, Chem. Comm. 36-38 and Holdgate et al., 2005, Drug Discov Today. The skilled person will know that commercial equipment is available for ΔG° measurements. ΔG° can also be estimated numerically by using the nearest neighbor model as described by Santa Lucia, 1998, Proc Natl Acad Sci USA. 95: 1460-1465 using appropriately derived thermodynamic parameters described by Sugimoto et al., 1995, Biochemistry 34:11211-11216 and McTigue et al., 2004, Biochemistry 43:5388-5405. In order to have the possibility of modulating its intended nucleic acid target by hybridization, oligonucleotides of the present invention hybridize to a target nucleic acid with estimated AG° values below −10 kcal for oligonucleotides that are 10-30 nucleotides in length. In some embodiments the degree or strength of hybridization is measured by the standard state Gibbs free energy ΔG° . The oligonucleotides may hybridize to a target nucleic acid with estimated ΔG° values below the range of −10 kcal, such as below −15 kcal, such as below −20 kcal and such as below −25 kcal for oligonucleotides that are 8-30 nucleotides in length. In some embodiments the oligonucleotides hybridize to a target nucleic acid with an estimated ΔG° value of −10 to −60 kcal, such as −12 to −40, such as from −15 to −30 kcal or −16 to −27 kcal such as −18 to −25 kcal.

As above, the invention relates to the use of a compound of formula (I):

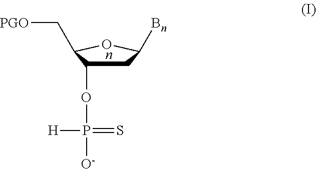

for the preparation of an oligonucleotide comprising at least one phosphorodithioate internucleoside linkage.

In Formula (I) PG is a hydroxyl-protecting group, Bn is a nucleobase that can be natural or non-natural, and ribose n is modified at the 2′ position of its ring.

### Hydroxyl-protecting Group PG

“Hydroxyl protecting group” PG is a protecting group of the hydroxyl group. Examples of hydroxyl protecting groups are acetyl (Ac), benzoyl (Bz), benzyl (Bn), β-methoxyethoxymethyl ether (MEM), dimethoxytrityl (or bis-(4-methoxyphenyl)phenylmethyl) (DMT), trimethoxytrityl (or tris-(4-methoxyphenyl)phenylmethyl) (TMT), methoxymethyl ether (MOM), methoxytrityl [(4-methoxyphenyl)diphenylmethyl (MMT), p-methoxybenzyl ether (PMB), methylthiomethyl ether, pivaloyl (Piv), tetrahydropyranyl (THP), tetrahydrofuran (THF), trityl or triphenylmethyl (Tr), silyl ether (for example trimethylsilyl (TMS), tert-butyldimethylsilyl (TBDMS), tri-iso-propylsilyloxymethyl (TOM) and triisopropylsilyl (TIPS) ethers), methyl ethers and ethoxyethyl ethers (EE). Particular examples of hydroxyl protecting group are DMT and TMT, in particular DMT.

### Nucleobase Bn

The term nucleobase includes the purine (e.g. adenine and guanine) and pyrimidine (e.g. uracil, thymine and cytosine) moieties present in nucleosides and nucleotides which form hydrogen bonds in nucleic acid hybridization. In the context of the present invention the term nucleobase also encompasses modified nucleobases which may differ from naturally occurring nucleobases, but are functional during nucleic acid hybridization. In this context “nucleobase” refers to both naturally occurring nucleobases such as adenine, guanine, cytosine, thymidine, uracil, xanthine and hypoxanthine, as well as non-naturally occurring variants. Such variants are for example described in Hirao et al (2012) Accounts of Chemical Research vol 45 page 2055 and Bergstrom (2009) Current Protocols in Nucleic Acid Chemistry Suppl. 37 1.4.1. (The nomenclature “Bn” indicates a particular nucleobase “n”, not that the moiety B is repeated n times).

In some embodiments the nucleobase moiety is modified by changing the purine or pyrimidine into a modified purine or pyrimidine, such as substituted purine or substituted pyrimidine, such as a nucleobase selected from isocytosine, pseudoisocytosine, 5-methyl cytosine, 5-thiozolo-cytosine, 5-propynyl-cytosine, 5-propynyl-uracil, 5-bromouracil 5-thiazolo-uracil, 2-thio-uracil, 2′thio-thymine, inosine, diaminopurine, 6-aminopurine, 2-aminopurine, 2,6-diaminopurine and 2-chloro-6-aminopurine.

The nucleobase moieties may be indicated by the letter code for each corresponding nucleobase, e.g. A, T, G, C or U, wherein each letter may optionally include modified nucleobases of equivalent function. For example, in the exemplified oligonucleotides, the nucleobase moieties are selected from A, T, G, C, and 5-methyl cytosine. Optionally, for LNA gapmers, 5-methyl cytosine LNA nucleosides may be used.

### Nucleoside Modifications

The ribose n in formula (I) is modified at the 2′ position of its ring, i.e. modified when compared to the ribose sugar moiety found in DNA and RNA.

In particular, the modification at the 2′ position of ribose n is selected from the group consisting of locked nucleic acid (LNA), constrained ethyl (cET), 2′-O-methoxyethyl (2′-O-MOE), 2′-O-Methyl and 2′-fluoro modifications, preferably LNA modification.

Numerous nucleosides with modification of the ribose moiety have been made, primarily with the aim of improving certain properties of oligonucleotides, such as affinity and/or nuclease resistance.

Such modifications include those where the ribose ring structure is modified, e.g. by replacement with a hexose ring (HNA), or a bicyclic ring, which typically have a biradical bridge between the C2 and C4 carbons on the ribose ring (LNA), or an unlinked ribose ring which typically lacks a bond between the C2 and C3 carbons (e.g. UNA). Other sugar modified nucleosides include, for example, bicyclohexose nucleic acids (WO 2011/017521) or tricyclic nucleic acids (WO 2013/154798). Modified nucleosides also include nucleosides where the sugar moiety is replaced with a non-sugar moiety, for example in the case of peptide nucleic acids (PNA), or morpholino nucleic acids.

Sugar modifications also include modifications made via altering the substituent groups on the ribose ring to groups other than hydrogen, or the 2′-OH group naturally found in DNA and RNA nucleosides. Substituents may, for example be introduced at the 2′, 3′, 4′ or 5′ positions.

### 2′ Modified Riboses

A 2′-modified ribose is a ribose which has a substituent other than H or —OH at the 2′ position (2′ substituted ribose) or comprises a 2′ linked biradical capable of forming a bridge between the 2′ carbon and a second carbon in the ribose ring, known as LNA (2′-4′ biradical bridged) nucleosides.

Indeed, much focus has been spent on developing 2′ substituted nucleosides, and numerous 2′ substituted nucleosides have been found to have beneficial properties when incorporated into oligonucleotides. For example, a 2′ modified ribose may provide enhanced binding affinity and/or increased nuclease resistance to the oligonucleotide. Examples of 2′ substituted modified nucleosides are 2′-O-alkyl-RNA, 2′-O-methyl-RNA, 2′-alkoxy-RNA, 2′-O-methoxyethyl-RNA (MOE), 2′-amino-DNA, 2′-fluoro-RNA and 2′-F-ANA nucleoside. Further examples can be found in e.g. Freier & Altmann; Nucl. Acid Res., 1997, 25, 4429-4443 and Uhlmann; Curr. Opinion in Drug Development, 2000, 3(2), 293-213 and Deleavey and Damha, Chemistry and Biology 2012, 19, 937. Below are illustrations of some nucleosides having 2′ substituted riboses.

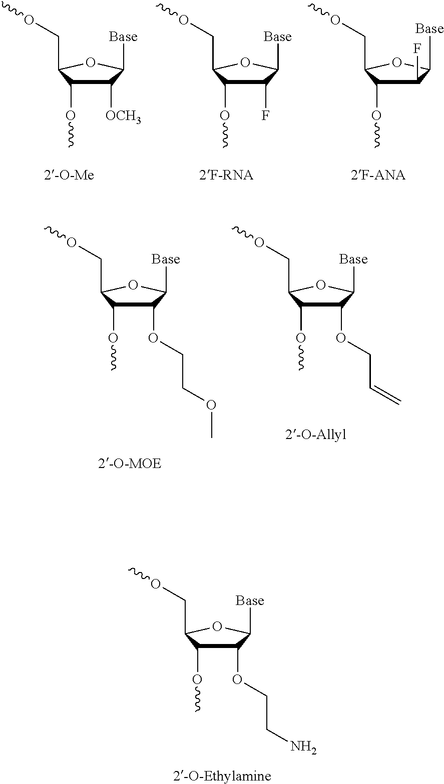

In relation to the present invention the term “modified at the 2′ position of the ribose ring” includes 2′ bridged molecules like LNA.

### Locked Nucleic Acid Nucleosides (LNA nucleosides)

A “LNA nucleoside” is a 2′-modified nucleoside which comprises a biradical linking the C2′ and C4′ of the ribose sugar ring of said nucleoside (also referred to as a “2′-4′ bridge”), which restricts or locks the conformation of the ribose ring. These nucleosides are also termed bridged nucleic acid or bicyclic nucleic acid (BNA) in the literature. The locking of the conformation of the ribose is associated with an enhanced affinity of hybridization (duplex stabilization) when the LNA is incorporated into an oligonucleotide for a complementary RNA or DNA molecule. This can be routinely determined by measuring the melting temperature of the oligonucleotide/complement duplex.

Non limiting, exemplary LNA nucleosides are disclosed in WO 99/014226, WO 00/66604, WO 98/039352, WO 2004/046160, WO 00/047599, WO 2007/134181, WO 2010/077578, WO 2010/036698, WO 2007/090071, WO 2009/006478, WO 2011/156202, WO 2008/154401, WO 2009/067647, WO 2008/150729, Morita et al., Bioorganic & Med.Chem. Lett. 12, 73-76, Seth et al. J. Org. Chem. 2010, Vol 75(5) pp. 1569-81 and Mitsuoka et al., Nucleic Acids Research 2009, 37(4), 1225-1238.

The 2′-4′ bridge in the LNA of the present invention may comprise 2 to 4 bridging atoms and is in particular of formula —X—Y—, in which X being linked to C4′ and Y linked to C2′, wherein

X is oxygen, sulfur, —CR^{a}R^{b}—, —C(R^{a})═C(R^{b})—, —C(═CR^{a}R^{b})—, —C(R^{a})═N—, —Si(R^{a})₂—, —SO₂—, —NR^{a}—; —O—NR^{a}—, —NR^{a}—O—, —C(═J)—, Se, —O—NR^{a}—, —NR^{a}—CR^{a}R^{b}—, —N(R^{a})—O— or —O—CR^{a}R^{b}—;

Y is oxygen, sulfur, —(CR^{a}R^{b})ₙ—, —CR^{a}R^{b}—O—CR^{a}R^{b}—, —C(R^{a})═C(R^{b})—, —C(R^{a})═N—, —Si(R^{a})₂—, —SO₂—, —NR^{a}—, —C(═J)—, Se, —O—NR^{a}—, —NR^{a}—CR^{a}R^{b}—, —N(R^{a})—O— or —O—CR^{a}R^{b}—;

with the proviso that —X—Y— is not —O—O—, Si(R^{a})₂—Si(R^{a})₂—, —SO₂—SO₂—, —C(R^{a})═C(R^{b})—C(R^{a})═C(R^{b}), —C(R^{a})═N—C(R^{a})═N—, —C(R^{a})═N—C(R^{a})═C(R^{b}), —C(R^{a})═C(R^{b})—C(R^{a})═N— or —Se—Se—;

J is oxygen, sulfur, ═CH₂ or ═N(R^{a});

R^{a} and R^{b} are independently selected from hydrogen, halogen, hydroxyl, cyano, thiohydroxyl, alkyl, substituted alkyl, alkenyl, substituted alkenyl, alkynyl, substituted alkynyl, alkoxy, substituted alkoxy, alkoxyalkyl, alkenyloxy, carboxyl, alkoxycarbonyl, alkylcarbonyl, formyl, aryl, heterocyclyl, amino, alkylamino, carbamoyl, alkylaminocarbonyl, aminoalkylaminocarbonyl, alkylaminoalkylaminocarbonyl, alkylcarbonylamino, carbamido, alkanoyloxy, sulfonyl, alkylsulfonyloxy, nitro, azido, thiohydroxylsulfidealkylsulfanyl, aryloxycarbonyl, aryloxy, arylcarbonyl, heteroaryl, heteroaryloxycarbonyl, heteroaryloxy, heteroarylcarbonyl, —OC(═X^{a})R^{c}, —OC(═X^{a})NR^{c}R^{d} and —NR^{e}C(═X^{a})NR^{c}R^{d};

or two geminal R^{a} and R^{b} together form optionally substituted methylene;

or two geminal R^{a} and R^{b}, together with the carbon atom to which they are attached, form cycloalkyl or halocycloalkyl, with only one carbon atom of —X—Y—;

wherein substituted alkyl, substituted alkenyl, substituted alkynyl, substituted alkoxy and substituted methylene are alkyl, alkenyl, alkynyl and methylene substituted with 1 to 3 substituents independently selected from halogen, hydroxyl, alkyl, alkenyl, alkynyl, alkoxy, alkoxyalkyl, alkenyloxy, carboxyl, alkoxycarbonyl, alkylcarbonyl, formyl, heterocylyl, aryl and heteroaryl;

X^{a} is oxygen, sulfur or —NR^{c};

R^{c}, R^{d} and R^{e} are independently selected from hydrogen and alkyl; and

n is 1, 2 or 3.

In a further particular embodiment of the invention, X is oxygen, sulfur, —NR^{a}—, —CR^{a}R^{b}— or —C(═CR^{a}R^{b})—, particularly oxygen, sulfur, —NH—, —CH₂— or —C(═CH₂)—, more particularly oxygen.

In another particular embodiment of the invention, Y is —CR^{a}R^{b}—, —CR^{a}R^{b}—CR^{a}R^{b}—or —CR^{a}R^{b}—CR^{a}R^{b}— CR^{a}R^{b}—, particularly —CH₂—CHCH₃—, —CHCH₃—CH₂—, —CH₂—CH₂— or —CH₂—CH₂—CH₂—.

In a particular embodiment of the invention, —X—Y— is —O—(CR^{a}R^{b})ₙ—, —S—CR^{a}R^{b}—, —N(R^{a})CR^{a}R^{b}—, —CR^{a}R^{b}—CR^{a}R^{b}—, —O—CR^{a}R^{b}—O—CR^{a}R^{b}—, —CR^{a}R^{b}—O—CR^{a}R^{b}—, —C(═CR^{a}R^{b})—CR^{a}R^{b}—, —N(R^{a})CR^{a}R^{b}—, —O—N(R^{a})—CR^{a}R^{b}— or —N(R^{a})—O—CR^{a}R^{b}—.

In a particular embodiment of the invention, R^{a} and R^{b} are independently selected from the group consisting of hydrogen, halogen, hydroxyl, alkyl and alkoxyalkyl, in particular hydrogen, halogen, alkyl and alkoxyalkyl.

In another embodiment of the invention, R^{a} and R^{b} are independently selected from the group consisting of hydrogen, fluoro, hydroxyl, methyl and —CH₂—O—CH₃, in particular hydrogen, fluoro, methyl and —CH₂—O—CH₃.

Advantageously, one of R^{a} and R^{b} of —X—Y— is as defined above and the other ones are all hydrogen at the same time.

In a further particular embodiment of the invention, R^{a} is hydrogen or alkyl, in particular hydrogen or methyl.

In another particular embodiment of the invention, R^{b} is hydrogen or or alkyl, in particular hydrogen or methyl.

In a particular embodiment of the invention, one or both of R^{a} and R^{b} are hydrogen.

In a particular embodiment of the invention, only one of R^{a} and R^{b} is hydrogen.

In one particular embodiment of the invention, one of R^{a} and R^{b} is methyl and the other one is hydrogen.

In a particular embodiment of the invention, R^{a} and R^{b} are both methyl at the same time.

In a particular embodiment of the invention, —X—Y— is —O—CH₂—, —S—CH₂—, —S—CH(CH₃)—, —NH—CH₂—, —O—CH₂CH₂—, —O—CH(CH₂—O—CH₃)—, —O—CH(CH₂CH₃)—, —O—CH(CH₃)—, —O—CH₂—O—CH₂—, —O—CH₂—O—CH₂—, —CH₂—O—CH₂—, —C(═CH₂)CH₂—, —C(═CH₂)CH(CH₃)—, —N(OCH₃)CH₂— or —N(CH₃)CH₂—;

In a particular embodiment of the invention, —X—Y— is —O—CR^{a}R^{b}— wherein R^{a} and R^{b} are independently selected from the group consisting of hydrogen, alkyl and alkoxyalkyl, in particular hydrogen, methyl and —CH₂—O—CH₃.

In a particular embodiment, —X—Y— is —O—CH₂— or —O—CH(CH₃)—, particularly —O—CH₂—.

The 2′- 4′ bridge may be positioned either below the plane of the ribose ring (beta-D-configuration), or above the plane of the ring (alpha-L-configuration), as illustrated in formula (A) and formula (B) respectively.

The ribose rings in the LNA nucleosides according to the invention may in particular have formula (B1) or (B2)

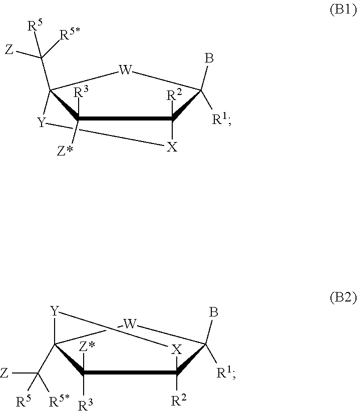

wherein

W is oxygen, sulfur, —N(R^{a})— or —CR^{a}R^{b}—, in particular oxygen;

B is a nucleobase or a modified nucleobase;

Z is an internucleoside linkage to an adjacent nucleoside or a 5′-terminal group;

Z* is an internucleoside linkage to an adjacent nucleoside or a 3′-terminal group;

R¹, R², R³, R⁵ and R^{5*} are independently selected from hydrogen, halogen, alkyl, haloalkyl, alkenyl, alkynyl, hydroxy, alkoxy, alkoxyalkyl, azido, alkenyloxy, carboxyl, alkoxycarbonyl, alkylcarbonyl, formyl and aryl; and

X, Y, R^{a} and R^{b} are as defined above.

In a particular embodiment, in the definition of —X—Y—, R^{a} is hydrogen or alkyl, in particular hydrogen or methyl. In another particular embodiment, in the definition of —X—Y—, R^{b} is hydrogen or alkyl, in particular hydrogen or methyl. In a further particular embodiment, in the definition of —X—Y—, one or both of R^{a} and R^{b} are hydrogen. In a particular embodiment, in the definition of —X—Y—, only one of R^{a} and R^{b} is hydrogen. In one particular embodiment, in the definition of —X—Y—, one of R^{a} and R^{b} is methyl and the other one is hydrogen. In a particular embodiment, in the definition of —X—Y—, R^{a} and R^{b} are both methyl at the same time.

In a further particular embodiment, in the definition of X, R^{a} is hydrogen or alkyl, in particular hydrogen or methyl. In another particular embodiment, in the definition of X, R^{b} is hydrogen or alkyl, in particular hydrogen or methyl. In a particular embodiment, in the definition of X, one or both of R^{a} and R^{b} are hydrogen. In a particular embodiment, in the definition of X, only one of R^{a} and R^{b} is hydrogen. In one particular embodiment, in the definition of X, one of R^{a} and R^{b} is methyl and the other one is hydrogen. In a particular embodiment, in the definition of X, R^{a} and R^{b} are both methyl at the same time.

In a further particular embodiment, in the definition of Y, R^{a} is hydrogen or alkyl, in particular hydrogen or methyl. In another particular embodiment, in the definition of Y, R^{b} is hydrogen or alkyl, in particular hydrogen or methyl. In a particular embodiment, in the definition of Y, one or both of R^{a} and R^{b} are hydrogen. In a particular embodiment, in the definition of Y, only one of R^{a} and R^{b} is hydrogen. In one particular embodiment, in the definition of Y, one of R^{a} and R^{b} is methyl and the other one is hydrogen. In a particular embodiment, in the definition of Y, R^{a} and R^{b} are both methyl at the same time.

In a particular embodiment of the invention R¹, R², R³, R⁵ and R^{5*} are independently selected from hydrogen and alkyl, in particular hydrogen and methyl.

In a further particular advantageous embodiment of the invention, R¹, R², R³, R⁵ and R^{5*} are all hydrogen at the same time.

In another particular embodiment of the invention, R¹, R², R³, are all hydrogen at the same time, one of R⁵ and R^{5*} is hydrogen and the other one is as defined above, in particular alkyl, more particularly methyl.

In a particular embodiment of the invention, R⁵ and R^{5*} are independently selected from hydrogen, halogen, alkyl, alkoxyalkyl and azido, in particular from hydrogen, fluoro, methyl, methoxyethyl and azido. In particular advantageous embodiments of the invention, one of R⁵ and R^{5*} is hydrogen and the other one is alkyl, in particular methyl, halogen, in particular fluoro, alkoxyalkyl, in particular methoxyethyl or azido; or R⁵ and R^{5*} are both hydrogen or both halogen at the same time, in particular both hydrogen or both fluoro at the same time. In such particular embodiments, W can advantageously be oxygen, and —X—Y— advantageously —O—CH₂—.

In a particular embodiment of the invention, —X—Y— is —O—CH₂—, W is oxygen and R¹, R², R³, R⁵ and R^{5*} are all hydrogen at the same time. Such LNA nucleosides are disclosed in WO 99/014226, WO 00/66604, WO 98/039352 and WO 2004/046160 which are all hereby incorporated by reference and include what are commonly known in the art as beta-D-oxy LNA and alpha-L-oxy LNA nucleosides.

In another particular embodiment of the invention, —X—Y— is —S—CH₂—, W is oxygen and R¹, R², R³, R⁵ and R^{5*} are all hydrogen at the same time. Such thio LNA nucleosides are disclosed in WO 99/014226 and WO 2004/046160 which are hereby incorporated by reference.

In another particular embodiment of the invention, —X—Y— is —NH—CH₂—, W is oxygen and R¹, R², R³, R⁵ and R^{5*} are all hydrogen at the same time. Such amino LNA nucleosides are disclosed in WO 99/014226 and WO 2004/046160 which are hereby incorporated by reference.

In another particular embodiment of the invention, —X—Y— is —O—CH₂CH₂— or —OCH₂CH₂ CH₂—, W is oxygen, and R¹, R², R³, R⁵ and R^{5*} are all hydrogen at the same time. Such LNA nucleosides are disclosed in WO 00/047599 and Morita et al., Bioorganic & Med.Chem. Lett. 12, 73-76, which are hereby incorporated by reference, and include what are commonly known in the art as 2′-O-4′C-ethylene bridged nucleic acids (ENA).

In another particular embodiment of the invention, —X—Y— is —O—CH₂—, W is oxygen, R¹, R², R³ are all hydrogen at the same time, one of R⁵ and R^{5*} is hydrogen and the other one is not hydrogen, such as alkyl, for example methyl. Such 5′ substituted LNA nucleosides are disclosed in WO 2007/134181 which is hereby incorporated by reference.

In another particular embodiment of the invention, —X—Y— is —O—CR^{a}R^{b}—, wherein one or both of R^{a} and R^{b} are not hydrogen, in particular alkyl such as methyl, W is oxygen, R¹, R², R³ are all hydrogen at the same time, one of R⁵ and R^{5*} is hydrogen and the other one is not hydrogen, in particular alkyl, for example methyl. Such bis modified LNA nucleosides are disclosed in WO 2010/077578 which is hereby incorporated by reference.

In another particular embodiment of the invention, —X—Y— is —O—CHR^{a}—, W is oxygen and R¹, R², R³, R⁵ and R^{5*} are all hydrogen at the same time. Such 6′-substituted LNA nucleosides are disclosed in WO 2010/036698 and WO 2007/090071 which are both hereby incorporated by reference. In such 6′-substituted LNA nucleosides, R^{a} is in particular C1-C6 alkyl, such as methyl.

In another particular embodiment of the invention, —X—Y— is —O—CH(CH2—O—CH3)- (“2′ O-methoxyethyl bicyclic nucleic acid”, Seth et al. J. Org. Chem. 2010, Vol 75(5) pp. 1569-81).

In another particular embodiment of the invention, —X—Y— is —O—CH(CH 2 CH 3)-;

In another particular embodiment of the invention, —X—Y— is —O—CH(CH2—O—CH3)—, W is oxygen and R¹, R², R³, R⁵ and R^{5*} are all hydrogen at the same time. Such LNA nucleosides are also known in the art as cyclic MOEs (cMOE) and are disclosed in WO 2007/090071.

In another particular embodiment of the invention, —X—Y— is —O—CH(CH₃)—(“2′O-ethyl bicyclic nucleic acid”, Seth at al., J. Org. Chem. 2010, Vol 75(5) pp. 1569-81).

In another particular embodiment of the invention, —X—Y— is —O—CH₂—O—CH₂— (Seth et al., J. Org. Chem 2010 op. cit.)

In another particular embodiment of the invention, —X—Y— is —O—CH(CH₃)-, W is oxygen and R¹, R², R³, R⁵ and R^{5*} are all hydrogen at the same time. Such 6′-methyl LNA nucleosides are also known in the art as cET nucleosides, and may be either (S)-cET or (R)-cET diastereoisomers, as disclosed in WO 2007/090071 (beta-D) and WO 2010/036698 (alpha-L) which are both hereby incorporated by reference.

In another particular embodiment of the invention, —X—Y— is —O—CR^{a}R^{b}—, wherein neither R^{a} nor R^{b} is hydrogen, W is oxygen and R¹, R², R³, R⁵ and R^{5*} are all hydrogen at the same time. In a particular embodiment, R^{a} and R^{b} are both alkyl at the same time, in particular both methyl at the same time. Such 6′-di-substituted LNA nucleosides are disclosed in WO 2009/006478 which is hereby incorporated by reference.

In another particular embodiment of the invention, —X—Y— is —S—CHR^{a}—, W is oxygen and R¹, R², R³, R⁵ and R^{5*} are all hydrogen at the same time. Such 6′-substituted thio LNA nucleosides are disclosed in WO 2011/156202 which is hereby incorporated by reference. In a particular embodiment of such 6′-substituted thio LNA, R^{a} is alkyl, in particular methyl.

In a particular embodiment of the invention, —X—Y— is —C(═CH₂)C(R^{a}R^{b})—, —C(═CHF)C(R^{a}R^{b})— or —C(═CF₂)C(R^{a}R^{b})—, W is oxygen and R¹, R², R³, R⁵ and R^{5*} are all hydrogen at the same time. R^{a} and R^{b} are advantageously independently selected from hydrogen, halogen, alkyl and alkoxyalkyl, in particular hydrogen, methyl, fluoro and methoxymethyl. R^{a} and R^{b} are in particular both hydrogen or methyl at the same time or one of R^{a} and R^{b} is hydrogen and the other one is methyl. Such vinyl carbo LNA nucleosides are disclosed in WO 2008/154401 and WO 2009/067647 which are both hereby incorporated by reference.

In a particular embodiment of the invention, —X—Y— is -N(OR^{a})-CH₂—, W is oxygen and R¹, R², R³, R⁵ and R^{5*} are all hydrogen at the same time. In a particular embodiment, R^{a} is alkyl such as methyl. Such LNA nucleosides are also known as N substituted LNAs and are disclosed in WO 2008/150729 which is hereby incorporated by reference.

In a particular embodiment of the invention, —X—Y— is —O—N(R^{a})—, —N(R^{a})—O—, —NR^{a}—CR^{a}R^{b}—CR^{a}R^{b}— or —NR^{a}-CR^{a}R^{b}—, W is oxygen and R¹, R², R³, R⁵ and R^{5*} are all hydrogen at the same time. R^{a} and R^{b} are advantageously independently selected from hydrogen, halogen, alkyl and alkoxyalkyl, in particular hydrogen, methyl, fluoro and methoxymethyl. In a particular embodiment, R^{a} is alkyl, such as methyl, R^{b} is hydrogen or methyl, in particular hydrogen. (Seth et al., J. Org. Chem 2010 op. cit.).

In a particular embodiment of the invention, —X—Y— is —O—N(CH₃)— (Seth et al., J. Org. Chem 2010 op. cit.).

In a particular embodiment of the invention, R⁵ and R^{5*} are both hydrogen at the same time. In another particular embodiment of the invention, one of R⁵ and R^{5*} is hydrogen and the other one is alkyl, such as methyl. In such embodiments, R¹, R² and R³ can be in particular hydrogen and —X—Y— can be in particular —O—CH₂— or —O—CHC(R^{a})₃—, such as —O—CH(CH₃)—.

In a particular embodiment of the invention, —X—Y— is -CR^{a}R^{b}—O—CR^{a}R^{b}—, such as —CH₂—O—CH₂—, W is oxygen and R¹, R², R³, R⁵ R^{5*} are all hydrogen at the same time. In such particular embodiments, R^{a} can be in particular alkyl such as methyl, R^{b} hydrogen or methyl, in particular hydrogen. Such LNA nucleosides are also known as conformationally restricted nucleotides (CRNs) and are disclosed in WO 2013/036868 which is hereby incorporated by reference.

In a particular embodiment of the invention, —X—Y— is —O—CR^{a}R^{b}—O—CR^{a}R^{b}—, such as —O—CH₂—O—CH₂—, W is oxygen and R¹, R², R³, R⁵ and R^{5*} are all hydrogen at the same time. R^{a} and R^{b} are advantageously independently selected from hydrogen, halogen, alkyl and alkoxyalkyl, in particular hydrogen, methyl, fluoro and methoxymethyl. In such a particular embodiment, R^{a} can be in particular alkyl such as methyl, R^{b} hydrogen or methyl, in particular hydrogen. Such LNA nucleosides are also known as COC nucleotides and are disclosed in Mitsuoka et al., Nucleic Acids Research 2009, 37(4), 1225-1238, which is hereby incorporated by reference.

It will be recognized than, unless otherwise specified, the riboses of the LNA nucleosides may be in the beta-D or alpha-L stereoisoform.

Particular examples of LNA nucleosides comprising 2′ modified riboses are presented in Scheme 1 (wherein B is as defined above).

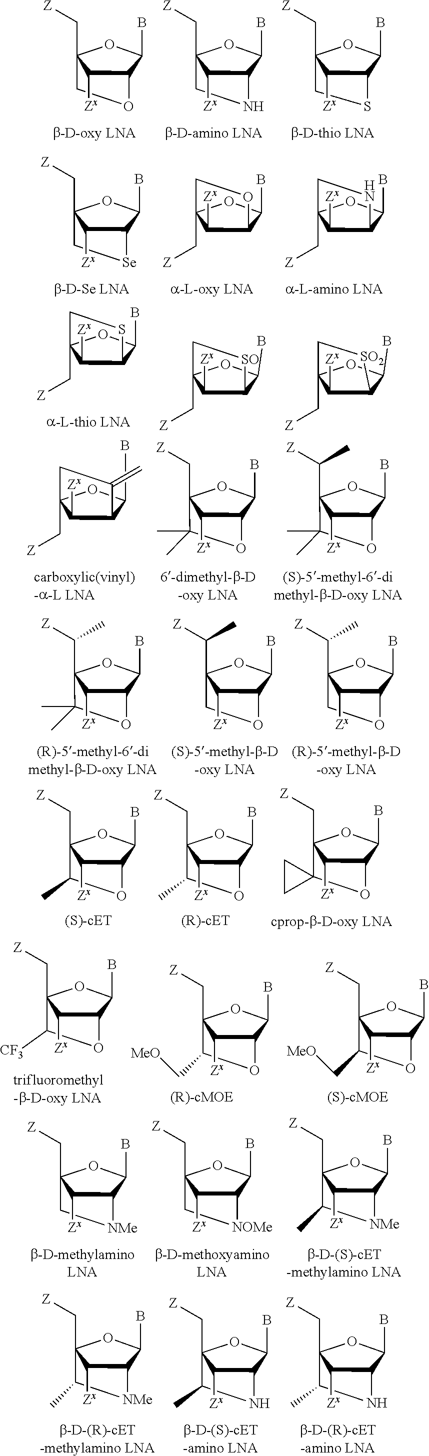

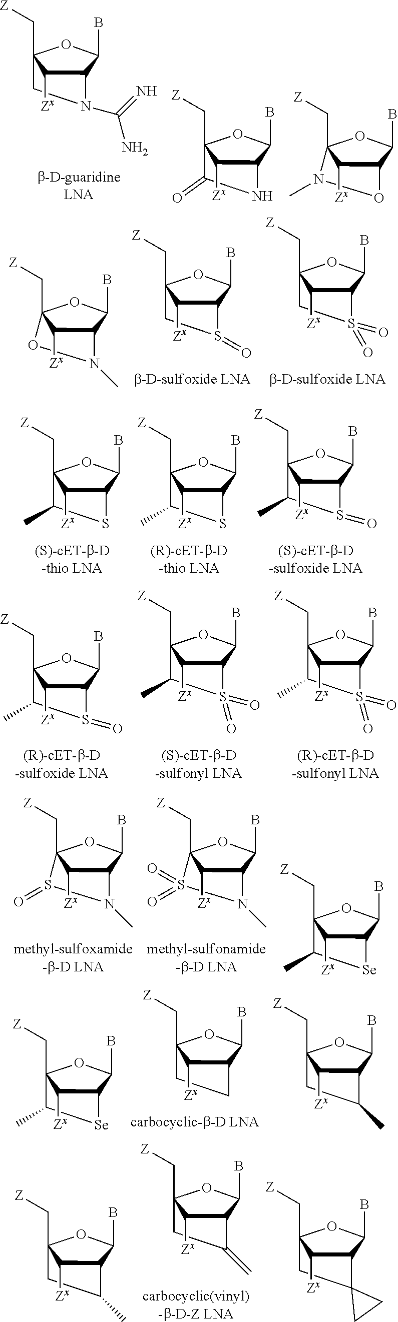

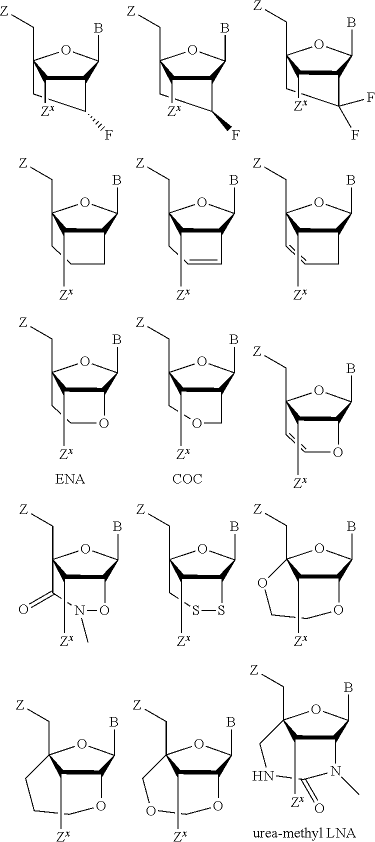

Particular LNA nucleosides are beta-D-oxy-LNA, 6′-methyl-beta-D-oxy LNA such as (S)-6′-methyl-beta-D-oxy-LNA ((S)-cET) and ENA.

### MOE Nucleoside

The term “MOE” stands for “methoxy-ethyl” and refers by means of abbreviation to a nucleoside having a ribose which is substituted at the 2′ position with a methoxy-ethoxy group as represented below.

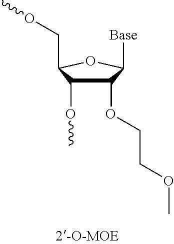

The above nucleoside can thus be named either “MOE” or “2′-O-MOE nucleoside”.

The invention also relates to a method for the preparation of an oligonucleotide containing at least one phosphorodithioate internucleoside linkage, said method comprising the steps of:
a) coupling a compound of formula (I):

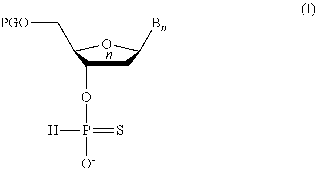

wherein—in formula (I)—PG is a hydroxyl-protecting group, Bn is a nucleobase that can be natural or non-natural, and wherein the ribose n is modified at the 2′ position of its ring, with a compound of formula (II):

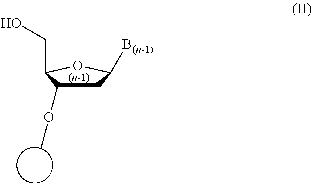

wherein—in formula (II)—B(n−1) is a nucleobase that can be natural or non-natural, and wherein ribose (n−1) can be unmodified or modified at the 2′ position of its ring, in the presence of a coupling agent, to obtain a compound of formula (III):

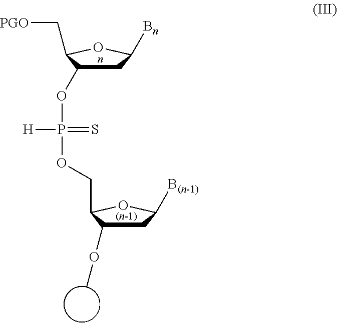

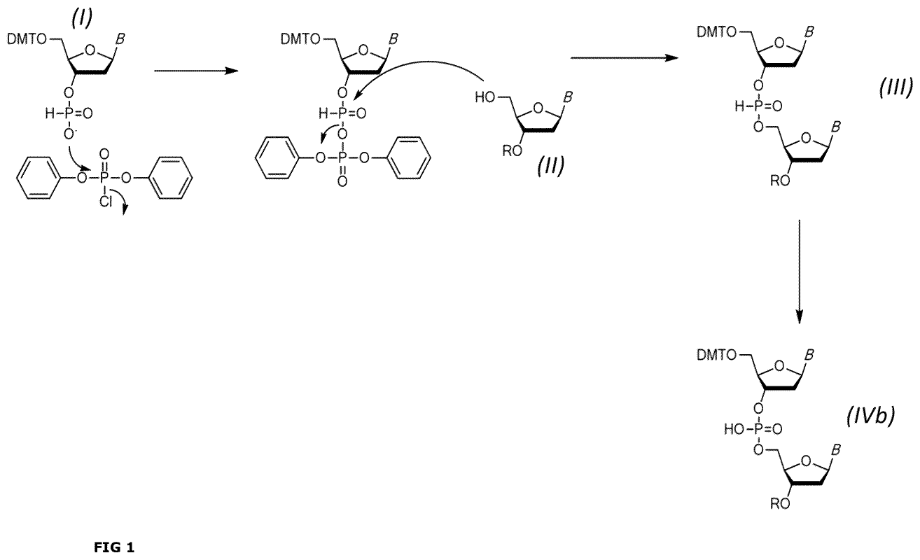

Details of the compound of formula (I) are as provided above. This method is illustrated in FIG. 1.

The compound of formula (II) is bonded to a solid support (illustrated in Formula (II) by the grey bead), making the method suitable for use in a high throughput oligonucleotide synthesizer. The compound of formula (II) is typically bonded to the solid support via a linker, preferably a cleavable linker. One or more additional nucleotides may also be present between nucleotide denoted ( n−1) and the solid phase. At the end of the oligonucleotide synthesis, the oligonucleotide is cleaved from the solid support. Suitable solid supports and linkers for use in the methods of the present invention are known to the skilled person.

All details of the nucleobase B(n−1) in formula (II) are as per nucleobase Bn in formula (I) above. Nucleobase B(n−1) may be the same or different to nucleobase Bn.

All details of the ribose (n−1) in formula (II) are as per ribose n in formula (I) above, apart from the fact that ribose (n−1) may be modified or unmodified at the 2′ position of its ring. If ribose (n−1) is modified at the 2′ position of its ring, such modification may be of the same type as described above for ribose n. If “unmodified” at the 2′ position of the ring, the ribose is either 2-OH ribose, or 2-deoxyribose.

Coupling of the compound of formula (I) with the compound of formula (II) takes place in the presence of a coupling agent. The coupling agent promotes the formation of the phosphoester linkage in formula (III). Preferably, the coupling agent is a diaryl chlorophosphate or a dialkyl chlorophosphate, e.g. diethyl chlorophosphate.

The method may further comprise the steps of:
b) performing a thioalkylation of the compound of formula (III) at the hydrogen bound to the phosphorus atom, to obtain a compound of formula (IVa):

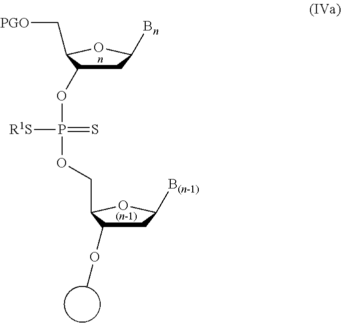

wherein R¹ is a cyanoalkyl, and wherein PG, Bn, B(n−1) and the riboses n and (n−1) are as defined for Formula (III);
c) or performing a reaction of the compound of formula (III) with a xanthane hydride to obtain a compound of formula (IVb):

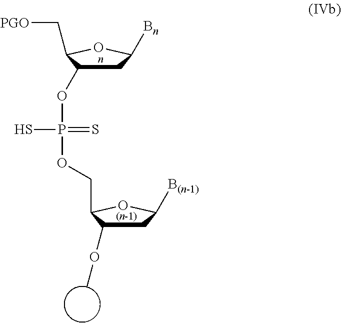

wherein PG, Bn, B(n−1) and the riboses n and (n−1) are as defined for Formula (III).

The method may further comprise the step of:
d) cleaving and deprotecting the compound of formula (IVa) or (IVb) to obtain a compound of formula (V):

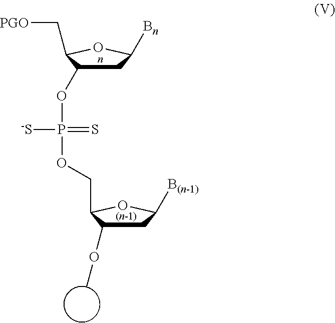

wherein PG, Bn, B(n−1) and the riboses n and (n−1) are as defined for Formula (III).

The method may further comprise the steps of:
deprotecting the compound of formula (IVa) to provide a compound of formula (Vla),

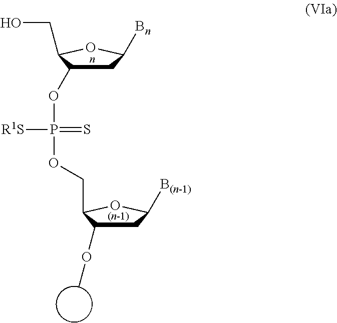

wherein Bn, B(n−1) and the riboses n and (n−1) are as defined for Formula (III);
followed by coupling a compound of formula (I) as defined above, or a 5′-O-protected ribonucleoside phosphoramidite, to the deprotected C5′-OH of the compound of formula (Vla) in the presence of a coupling agent; or
deprotecting the compound of formula (IVb) to provide a compound of formula (Vlb),

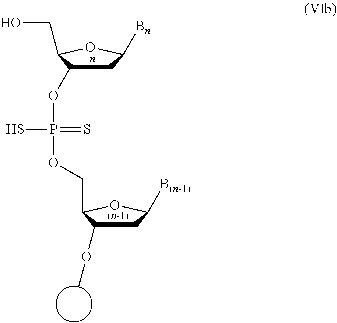

wherein Bn, B(n−1) and the riboses n and (n−1) are as defined for Formula (III);

followed by coupling a compound of formula (I) as defined above, or a 5′—O—protected ribonucleoside phosphoramidite, to the deprotected C5′—OH of the compound of formula (Vlb) in the presence of a coupling agent; or,

deprotecting the compound of formula (III), to provide a compound of formula

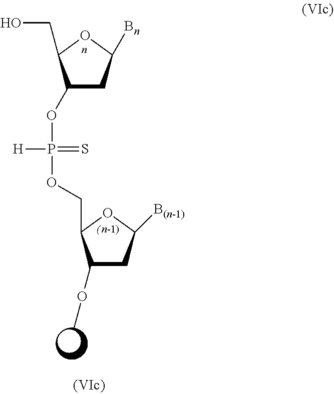

wherein Bn, B(n−1) and the riboses n and (n−1) are as defined for Formula (III);

followed by coupling a compound of formula (I) as defined above, or a 5′-O-protected ribonucleoside phosphoramidite, to the deprotected C5′-OH of the compound of formula (Vic) in the presence of a coupling agent.

The method may further comprise the steps of:
e) performing a global sulfurization of the compound of formula (III), followed by steps of cleaving and deprotecting to obtain a compound of formula (V):

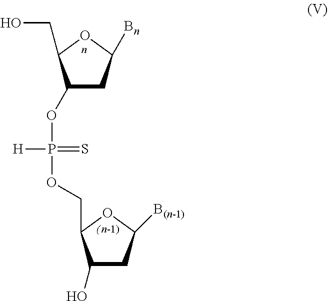

The coupling agent is suitably a dialkylchlorophosphate, preferably diethyl chlorophosphate, in combination with pyridine.

The invention also relates to a phosphonothioate monoester selected from the group consisting of:

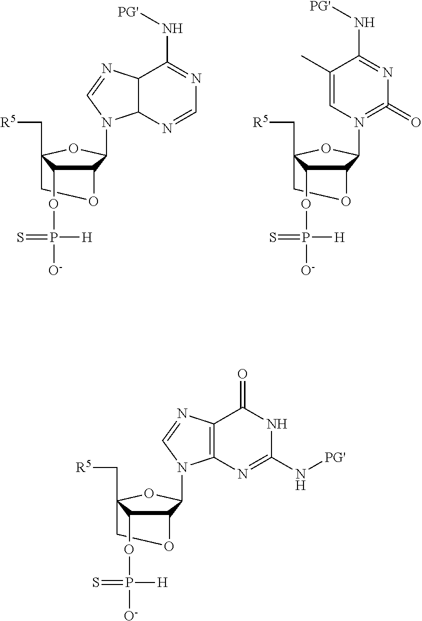

wherein PG′ is an amino-protecting group and R⁵ is H or a hydroxyl-protecting group.

In a particular embodiment, the invention also relates to a phosphonothioate monoester salt selected from the group consisting of:

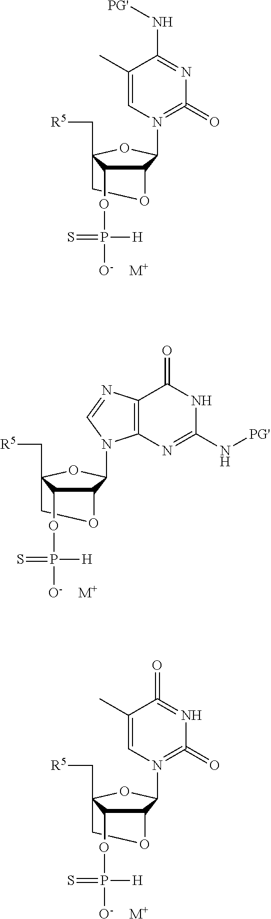

wherein PG′ is an amino-protecting group, M⁺ is a monovalent cation and R⁵ is H or a hydroxyl-protecting group.

M⁺ is a monovalent cation, such as a metal cation, e.g. an alkali metal cation, such as Na⁺ or K⁺, M⁺ may—in particular—be a quaternary ammonium cation, NR₄⁺ (in which R is H or C₁-C₄ alkyl).

### DETAILED DESCRIPTION OF THE FIGURES

In FIG. 1, a H-phosphonothioate monoester, being representative of a compound of formula (I), is activated by a coupling agent, usually an acid chloride such as pivaloyl chloride or diphenyl chlorophosphate. Diphenyl chlorophosphate has been shown to be O-selective.

The activated monoester is reacted (preferably immediately) with the desired alcohol being representative of a compound of formula (II). The H-phosphonothioate diester (representative of a compound of formula (III)) is relatively stable. The H-phosphonothionate diester can be converted into various P(V) compounds such a phosphorodithioate diester (using a sulfurizing agent) or a phosphorodithioate triester (using a thioalkylating agent).

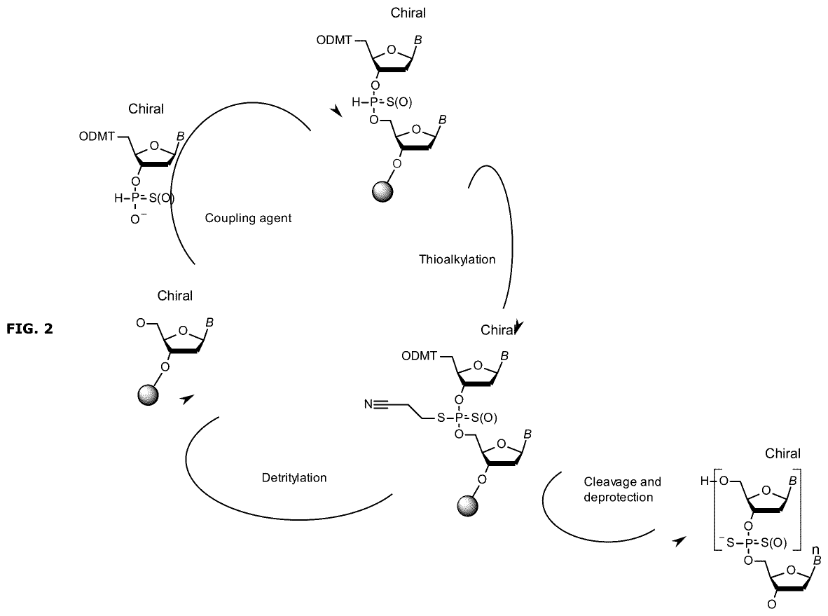

FIG. 2 shows oligomer synthesis using compounds of formula (I) with sulfurization (thioalkylation) after each coupling. If the H-phosphonothioate diester is converted into a (more stable) protected phosphoro(di)thioate (e.g. a cyanoethyl triester) the phosphonate chemistry can be combined with regular amidite chemistry in the same oligomer. In this way, an oligomer with phosphate, phosporothioate and phosphorodithioate linkages in the same oligomer is possible.

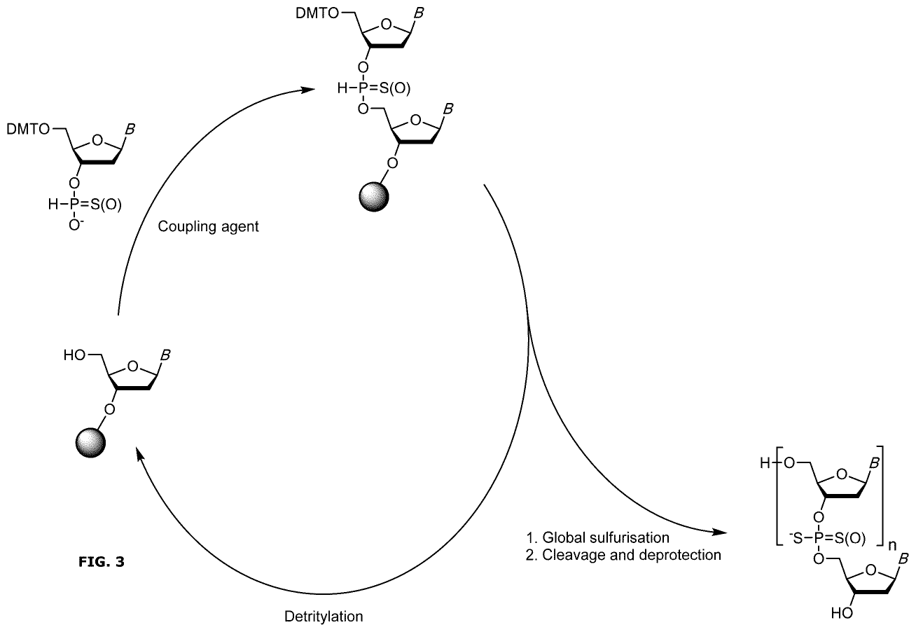

FIG. 3 illustrates oligomer synthesis using compounds of formula (I) with final sulfurization. If only H-phosphonates and/or H-phosphono(thio)ates are used in the synthesis cycles, the oxidation (sulfurization) step can be omitted after each coupling and instead be performed globally after the oligomer synthesis is finished. This provides to two types of phosphate linkages in the final chain, e.g. phosphorothioate/dithioate combinations.

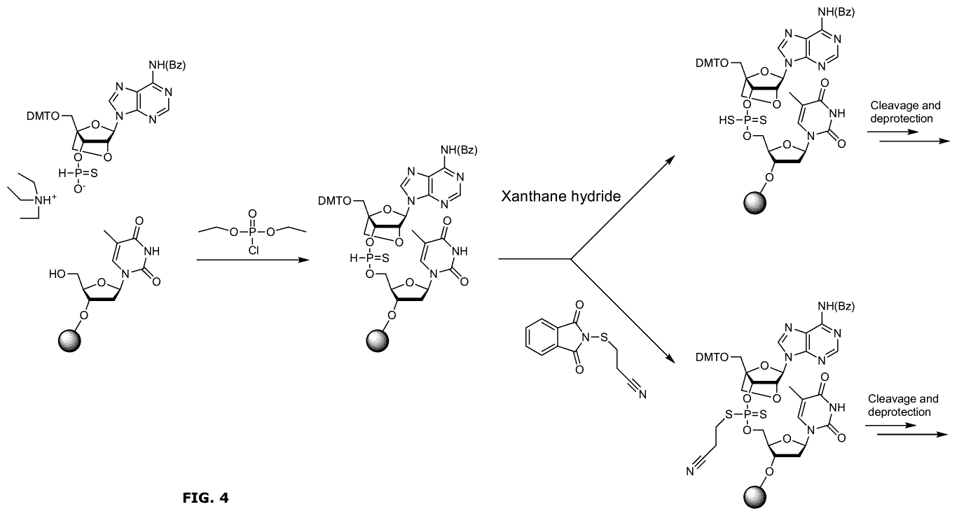

FIG. 4 shows a particular example of coupling of a H-phosphonothioate on a solid support. Conditions suitable for machine synthesis were developed first in solution and then verified on the machine. A combination of diethyl chlorophosphate with a limited amount of pyridine constitute the best conditions found so far. The diester was either sulfurised or thioalkylated before cleavage and protection.

The invention relates to the following numbered aspects:

Aspect 1. Use of a compound of formula (I):

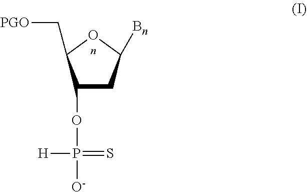

for the preparation of an oligonucleotide comprising at least one phosphorodithioate internucleoside linkage,
wherein PG is a hydroxyl-protecting group, Bn is a nucleobase that can be natural or non-natural, and wherein ribose n is modified at the 2′ position of its ring.

Aspect 2. The use according to aspect 1, wherein the modification at the 2′ position of ribose n is selected from the group consisting of locked nucleic acid (LNA), constrained ethyl (cET), 2′-O-methoxyethyl (2′-O-MOE), 2′-O-Methyl and 2′-fluoro modifications, preferably LNA modification.

Aspect 3. The use according to any one of the preceding aspects for the preparation of oligonucleotide comprising at least one phosphorodithioate and at least one phosphorothioate internucleoside linkage.

Aspect 4. The use according to any one of the preceding aspects for the preparation of an oligonucleotide comprising at least one phosphorodithioate, at least one phosphorothioate and/or at least one phosphodiester internucleoside linkage.

Aspect 5. A method for the preparation of an oligonucleotide containing at least one phosphorodithioate internucleoside linkage, said method comprising the steps of: b) coupling a compound of formula (I):

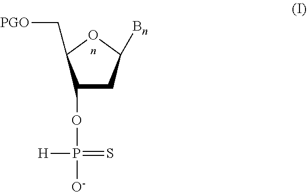

wherein—in formula (I)—PG is a hydroxyl-protecting group, Bn is a nucleobase that can be natural or non-natural, and wherein the ribose n is modified at the 2′ position of its ring, with a compound of formula (II):

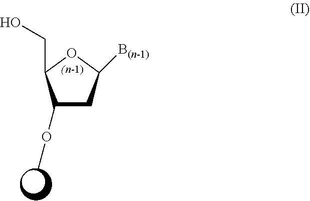

wherein—in formula (II)—B(n−1) is a nucleobase that can be natural or non-natural, and
wherein ribose (n−1) can be unmodified or modified at the 2′ position of its ring, in the presence of a coupling agent, to obtain a compound of formula (III):

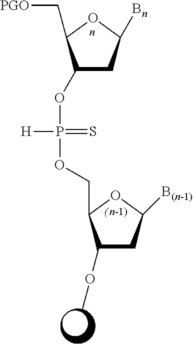

Aspect 6. The method according to aspect 5 further comprising the steps of:
f) performing a thioalkylation of the compound of formula (III) at the hydrogen bound to the phosphorus atom, to obtain a compound of formula (IVa):

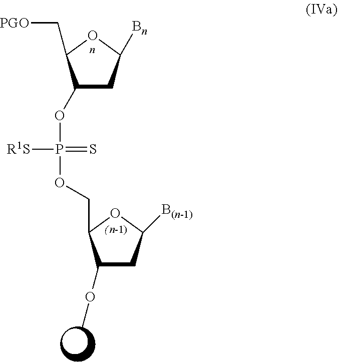

wherein R¹ is a cyanoalkyl, and wherein PG, Bn, B(n−1) and the riboses n and (n−1) are as defined for Formula (III);
g) or performing a reaction of the compound of formula (III) with a xanthane hydride to obtain a compound of formula (IVb):

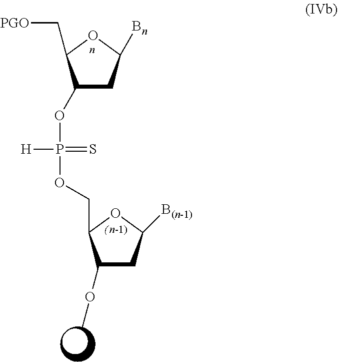

wherein PG, Bn, B(n−1) and the riboses n and (n−1) are as defined for Formula (III).

Aspect 7. The method according to aspect 6, further comprising the step of:
h) cleaving and deprotecting the compound of formula (IVa) or (IVb) to obtain a compound of formula (V):

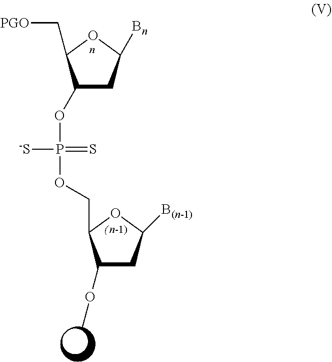

wherein PG, Bn, B(n−1) and the riboses n and (n−1) are as defined for Formula (III).

Aspect 8. The method according to aspect 6, further comprising the step of:
deprotecting the compound of formula (IVa) to provide a compound of formula (Vla),

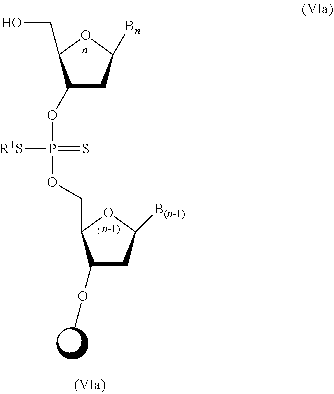

wherein Bn, B(n−1) and the riboses n and (n−1) are as defined for Formula (III);

followed by coupling a compound of formula (I) as defined above, or a 5′-O-protected ribonucleoside phosphoramidite, to the deprotected C5′-OH of the compound of formula (Via) in the presence of a coupling agent; or

deprotecting the compound of formula (IVb) to provide a compound of formula (Vlb),

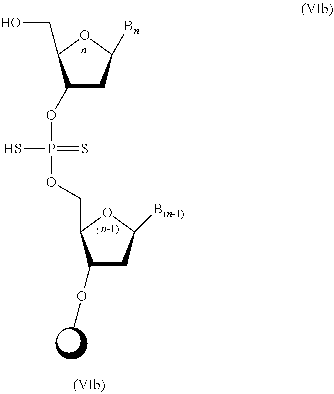

wherein Bn, B(n−1) and the riboses n and (n−1) are as defined for Formula (III);

followed by coupling a compound of formula (I) as defined above, or a 5′-O-protected ribonucleoside phosphoramidite, to the deprotected C5′-OH of the compound of formula (Vlb) in the presence of a coupling agent; or,

deprotecting the compound of formula (III), to provide a compound of formula

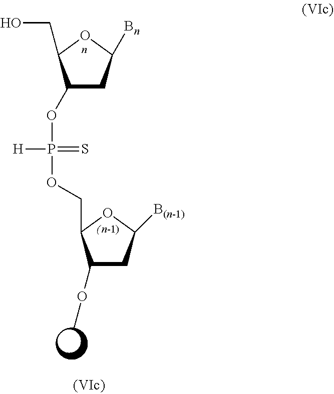

wherein Bn, B(n−1) and the riboses n and (n−1) are as defined for Formula (III);

followed by coupling a compound of formula (I) as defined above, or a 5′-O-protected ribonucleoside phosphoramidite, to the deprotected C5′-OH of the compound of formula (Vic) in the presence of a coupling agent.

Aspect 9. The method according to aspect 5, further comprising the step of:
i) performing a global sulfurization of the compound of formula (III), followed by steps of cleaving and deprotecting to obtain a compound of formula (V):

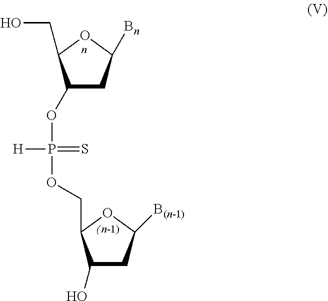

Aspect 10. The method of any one of aspects 2 to 9, wherein the coupling agent is a dialkylchlorophosphate, preferably diethyl chlorophosphate, in combination with pyridine.

Aspect 11. A phosphonothioate monoester selected from the group consisting of:

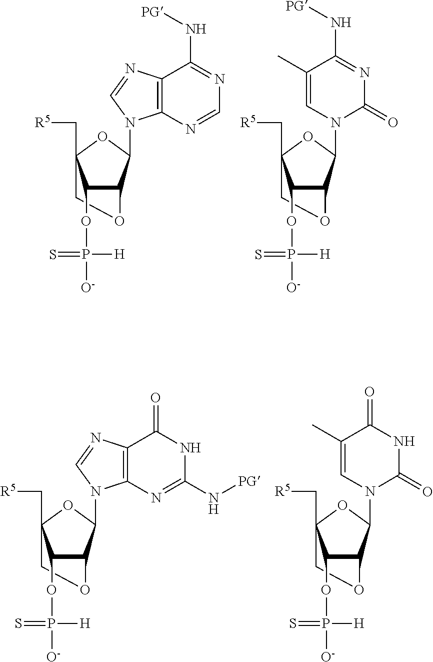

wherein PG′ is an amino-protecting group and R⁵ is H or a hydroxyl-protecting group.

Aspect 12. A phosphonothioate monoester salt selected from the group consisting of:

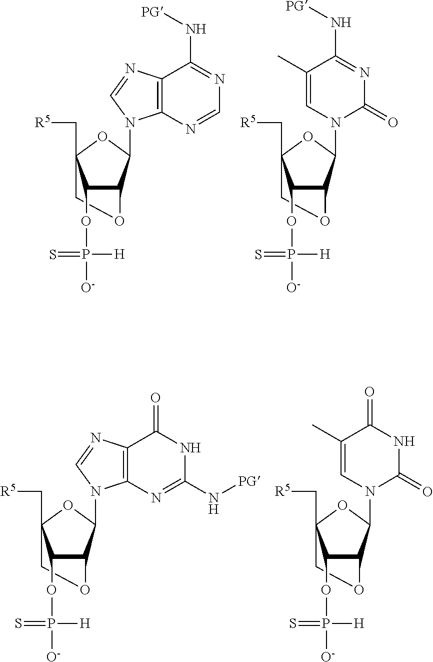

wherein PG′ is an amino-protecting group, M⁺ is a monovalent cation and R⁵ is H or a hydroxyl-protecting group.

### EXAMPLES

The invention will now be illustrated by the following examples which have no limiting character.

### DMTr-LNA-^{5Me}C^{Bz}-H-phosphonothioate, Triethyl Ammonium Salt

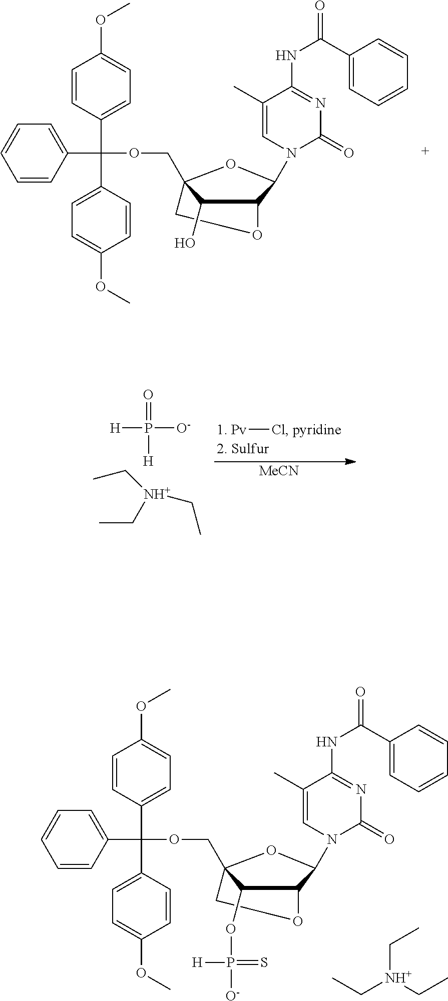

Triethylammonium phosphinate (1.9 g, 11.4 mmol) and DMTr-LNA-^{5-Me}C^{Bz} (9.97 g, 14.8 mmol, 1.3 14.8 mmol, 1.3 eqiuv.) was dried by co-evaporation from pyridine once. The residue was dissolved in acetonitrile (47.5 mL, 25 vol) and pyridine (9.5 mL, 5 vol). Solution was cooled on ice and pivaloyl chloride was added (1.6 mL, 1.15 equiv.). After stirring for 20 minutes, sulphur (727 mg, 2 equiv.) was added, and reaction stirred at rt for 1 hour. The reaction was quenched with 10 mL 1 M TEAB (triethylammonium bicarbonate) and evaporated. The residue was dissolved in 50 mL DCM and washed with 50 mL 1 M TEAB. The organic phase was evaporated and chromatographed on a 100 g Biotage HC silica column first with DCM:MeCN 3:1 to elute impurities and residual nucleoside followed by a MeOH (0-5%) in DCM to elute the product. Product fractions evaporated and dissolved in 50% MeOH (aq.) and applied to a 120 g Biotage C18 RP-column. Column was eluted with a 50-100% gradient of MeOH in water. Product eluting at ˜80% MeOH. Product fractions was pooled, concentrated to remove most of the methanol and extracted with 3*50 mL DCM, adding 1 M TEAB to aid separation. The combined organic phase was evaporated followed by a co-evaporation with MeCN. Yield 4.5 g of white foam (51% based on triethylammonium phosphinate).

³¹P-NMR (202 MHz, CD₃CN, δ in ppm) 53.32 and 52.50 (¹J_{PH}=578.0 and 571.5 Hz, d; ³J_{PH}=10.5 and 9.2 Hz, d); ¹H-NMR (500 MHz, CD₃CN, δ in ppm) (multiplicity of some signals due to the presence of P-diastereomers) 13.3 (br s, 1H), 11.2 (br s, 1H), 8.37-8.20 (br 2s, 2H), 7.95 and 7.94 (¹J_{PH}=578.0 and 571.5 Hz, 1H), 7.94 and 7.87 (2s, 1H), 7.63-7.25 (m, 12H), 6.96-6.90 (m, 4H), 5.63 (s, 1H), 5.06 and 4.87 (2d, J=10.6 and 9.20 Hz, 1H), 4.63 and 4.60 (2s, 1H), 3.93-3.81 (m, 2H), 3.80 (s, 6H), 3.61-3.48 (m, 2H), 3.01 (q, J=7.27 Hz, 6H), 1.87 and 1.86 (2s, 3H), 1.20 (t, J=7.25 Hz, 9H); LC-MS ESI (m/z) calcd for C₃₉H₃₇N₃O₉PS (M-TEAK⁺) 754.2, found 754.2

### DMTr-LNA-A^{Bz}-H-phosphonothioate, Triethyl Ammonium Salt

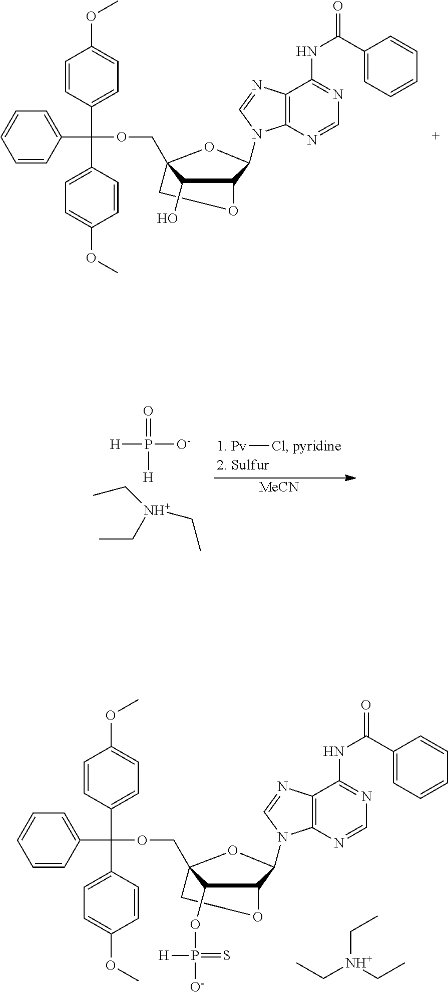

Triethylammonium phosphinate (1.74 g, 10.4 mmol) and DMTr-LNA-A^{Bz} (9.28 g, 13.5 mmol, 1.3 eqiuv.) was dried by co-evaporation from pyridine once. The residue was dissolved in acetonitrile (43.5 mL, 25 vol) and pyridine (8.7 mL, 5 vol). Solution was cooled on ice and pivaloyl chloride was added (1.46 mL, 1.15 equiv.). After stirring for 20 minutes, sulphur (666 mg, 2 equiv.) was added, and reaction stirred at rt for 1 hour. The reaction was quenched with 10 mL 1 M TEAB (triethylammonium bicarbonate) and evaporated. The residue was dissolved in 50 mL DCM and washed with 50 mL 1 M TEAB. The organic phase was evaporated and chromatographed on a 100 g Biotage HC silica column first with DCM:MeCN 3:1 to elute impurities and residual nucleoside followed by a MeOH (0-5%) in DCM to elute the product. Product fractions evaporated and dissolved in 50% MeOH (aq.) and applied to a 120 g Biotage C18 RP-column. Column was eluted with a 50-100% gradient of MeOH in water. Product eluting at ˜70% MeOH. Product fractions was pooled, concentrated to remove most of the methanol and extracted with 3*50 mL DCM, adding 1 M TEAB to aid separation. The combined organic phase was evaporated followed by a co-evaporation with MeCN. Yield 6.5 g of white foam (73% based on triethylammonium phosphinate).

³¹P-NMR (202 MHz, CD₃CN, δ in ppm) 53.43 and 52.50 (¹J_{PH}=579.4 and 574.8 Hz, d; ³J_{PH}=10.5 and 8.9 Hz, d); ¹H-NMR (500 MHz, CD₃CN, δ in ppm) (multiplicity of some signals due to the presence of P-diastereomers) 11.0 (br s, 1H), 9.51 (br s, 1H), 8.71 (br s, 1H), 8.59 and 8.50 (2s, 1H), 8.03 (br, 2H),),(¹J_{PH}=578.0 and 7.94 and 7.91 (¹J_{PH}=575.0 and 579 Hz, 1H), 7.67-7.21 (m, 12H), 6.91-6.85 (m, 4H), 6.16 and 6.15 (2s, 1H), 5.19 and 5.06 (2d, ¹J_{PH}=10.2 and 8.3 Hz, 1H), 4.94 and 4.91 (2s, 1H), 4.16-3.95 (m, 2H), 3.77 (s, 6H), 3.61-3.44 (m, 2H), 2.95 (q, ¹J_{PH}=7.3 Hz, 6H), 1.5 (t, ¹H_{PH}=7.3 Hz, 9H); LC-MS ESI (m/z) calcd for C₃₉H₃₅N₅O₈PS (M-TEAH⁺) 764.2, found 764.2

### DMTr-LNA-T-H-phosphonothioate, Triethyl Ammonium Salt

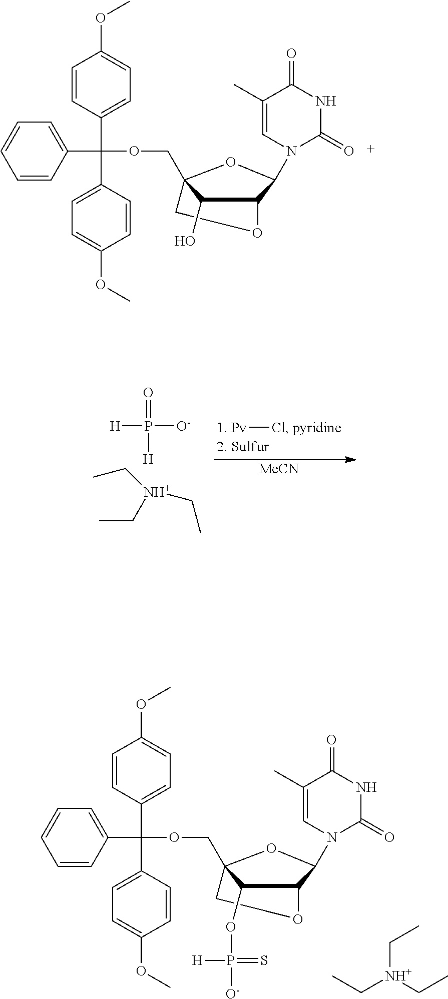

Triethylammonium phosphinate (1.9 g, 11.4 mmol) and DMTr-LNA-T (8.46 g, 14.8 mmol, 1.3 eqiuv.) was dried by co-evaporation from pyridine once. The residue was dissolved in acetonitrile (47.5 mL, 25 vol) and pyridine (9.5 mL, 5 vol). Solution was cooled on ice and pivaloyl chloride was added (1.6 mL, 1.15 equiv.). After stirring for 20 minutes, sulphur (727 mg, 2 equiv.) was added, and reaction stirred at rt for 1 hour. The reaction was quenched with 10 mL 1 M TEAB (triethylammonium bicarbonate) and evaporated. The residue was dissolved in 50 mL DCM and washed with 50 mL 1 M TEAB. The organic phase was evaporated and chromatographed on a 100 g Biotage HC silica column first with DCM:MeCN 3:1 to elute impurities and residual nucleoside followed by a Me0H (0-5%) in DCM to elute the product. Product fractions evaporated and dissolved in 40% MeOH (aq.) and applied to a 120 g Biotage C18 RP-column. Column was eluted with a 40-100% gradient of MeOH in water. Product eluting at ˜50% MeOH. Product fractions was pooled, concentrated to remove most of the methanol and extracted with 3*50 mL DCM, adding 1 M TEAB to aid separation. The combined organic phase was evaporated followed by a co-evaporation with MeCN. Yield 6.6 g of white foam (75% based on triethylammonium phosphinate).

³¹P-NMR (202 MHz, CD₃CN, δ in ppm) 53.38 and 53.06 (¹J_{PH}=573.3 and 578.3 Hz, d; ³J_{PH}=9.2 and 9.7 Hz, d); ¹H-NMR (500 MHz, CD₃CN, δ in ppm) (multiplicity of some signals due to the presence of P-diastereomers)10.8 (br s, 1H), 10.0 and 9.72 (2s, 1H), 8.04 and 7.95 (¹J_{PH}=573.0 and 578 Hz, 1H), 7.71-7.24 (m, 10H), 6.97-6.88 (m, 4H), 5.57 and 5.55 (2d, 1H), 5.02 and 4.90 (2d, ¹J_{PH}=9.8 and 9.2, 1H), 4.59 and 4.57 (2s, 1H), 3.89-3.80 (m, 2H), 3.79 (s, 6H), 3.57-3.41 (m, 2H), 3.05 (q, ¹J_{PH}=7.3 Hz, 6H), 1.66-1.63 (m, 3H), 1.21 (t, ¹J_{PH}=7.3 Hz, 9H); LC-MS ESI (m/z) calcd for C₃₂H₃₂N₂O₉PS (M-TEAK⁺) 651.7, found 652.2

### DMTr-LNA-G′ b -H-phosphonothioate, Triethylammonium Salt

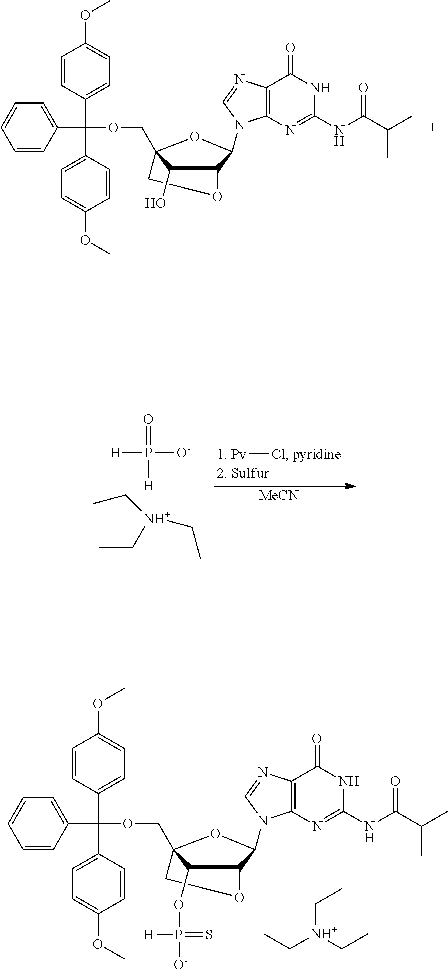

Triethylammonium phosphinate (1.9 g, 11.4 mmol) and DMTr-LNA-G^{1b} (9.86 g, 14.8 mmol, 1.3 eqiuv.) was dried by co-evaporation from pyridine. The residue was dissolved in acetonitrile (47.5 mL, 25 vol) and pyridine (9.5 mL, 5 vol). Solution was cooled on ice and pivaloyl chloride was added (1.6 mL, 1.15 equiv.). After stirring for 20 minutes, sulphur (727 mg, 2 equiv.) was added, and reaction stirred at rt for 1 hour. The reaction was quenched with 10 mL 1 M TEAB (triethylammonium bicarbonate) and evaporated. The residue was dissolved in 50 mL DCM and washed with 50 mL 1 M TEAB. The organic phase was evaporated and chromatographed on a 100 g Biotage HC silica column first with DCM:MeCN 3:1 to elute impurities and residual nucleoside followed by a MeOH (0-5%) in DCM to elute the product. Product fractions evaporated and dissolved in 50% MeOH (aq.) and applied to a 120 g Biotage C18 RP-column. Column was eluted with a 50-100% gradient of MeOH in water. Product eluting at ˜80% MeOH. Product fractions was pooled, concentrated to remove most of the methanol and extracted with 3*50 mL DCM, adding 1 M TEAB to aid separation. The combined organic phase was evaporated followed by a co-evaporation with MeCN. Yield 5.5 g of white foam (56% based on triethylammonium phosphinate).

³¹P-NMR (202 MHz, CD₃CN, δ in ppm) 52.18 and 51.98 (¹J_{PH}=576.9 and 572.2 Hz, d; ³J_{PH}=10.4 and 10.0 Hz, d); ¹H-NMR (500 MHz, CD₃CN, δ in ppm) (multiplicity of some signals due to the presence of P-diastereomers) 12.0 (br s, 1H), 11.0 and 10.8 (2s, 1H), 10.4 (br s, 1H), 7.99 and 7.96 (¹J_{PH}=572.0 and 577 Hz, 1H), 7.98 and 7.94 (2s, 1H), 7.49-7.20 (m, 9H), 6.90-6.84 (m, 4H), 5.86 (2s, 1H), 5.60 and 5.54 (2d, J=10.2 Hz, 1H), 4.87 (d, J=7.49 Hz, 1H), 4.11-3.96 (m, 2H), 3.77 (s, 6H), 3.47-3.40 (m, 2H), 3.04 (q, J=7.24 Hz, 6H), 2.84-2.71 (m, 1H), 1.23-1.17 (m, 15 h); LC-MS ESI (m/z) calcd for C₃₆H₃₇N₅O₉PS (M-TEAK⁺) 746.2, found 746.3

### Oligonucleotide Synthesis

Oligonucleotides were synthesised using a ÄKTA™ oligopilot™ plus 10 from Amersham Pharmacia Biotech (Cytiva). Synthesis were conducted on a 50 μmol scale in 1.2 mL columns using Nittophase HL Unylinker 400 having a loading of 405 μmol/g. Volume of recirculation loop was ˜2.5 mL.

Phosphorothioate and phosphate nucleotides were introduced using cyanoethyl amidite and standard template synthesis methods. Amidites were prepared as 0.1 M solutions in acetonitrile except LNA-MeC that was 0.1 M in acetonitrile/THF 3:1.

Phosphorodithioate nucleotides were introduced using 0.1 M H-phosphonothioate monoester solutions in acetonitrile containing 0.3 M pyridine. As coupling agent for H-phosphonothioate monoesters 0.2M diethyl chlorophosphate in acetonitrile was used. The thioalkylating agent 2-(2-cyanoethyl)sulfanyl-1H-isoindole-1,3-(2H)-dione was used as a 0.1 M solution in acetonitrile containing 0.5 M pyridine.

Detritylation was performed using 3% DCA in toluene. As amidite activator 0.3 M BTT (5-(Benzylthio)-1H-tetrazole) was used. Amidite couplings were performed using 3 equiv. of amidite and a 1.5:1 v/v ratio between activator and amidite, a recirculation time of 2.5 min for DNA and 5 min for LNA were employed. Sulfurisation of phosphite triester were performed with a 0.1 M solution of xanthane hydride (3-amino-1,2,4-dithiazole-5-thione) in acetonitrile/pyridine 1:1 using a 5 min contact time. Oxidation of phosphite triesters were performed using 0.05 M iodine in pyridine/water 9:1 using a 5 min contact time. Capping were performed with a 1/1 ratio of 20% N-methylimidazole in acetonitrile and 20% acetic anhydride and 30% lutidine in acetonitrile for 1 min.

For phosphorodithioate linkages the couplings were performed using 3 equiv. of monoester and a 1:1 v/v ratio of monoester/coupling agent and 6 min of recirculation. The H-phosphonothioate diesters were thioalkylated using 3 equiv. of thioalkylating agent and 3 min of recirculation.

The cyanoethyl groups were deprotected with 20% DEA in acetonitrile using 10 min flow through and 50 min recirculation.

Final cleavage from support and nucleobase deprotection was performed using concentrated aqueous ammonia for 15 hours at 55° C. The support was filtered and washed with water. Filtrate was analysed for OD measurement and area-% full length product, FLP, (DMT-on, at 260 nm). The amount of des-sulfur (0 instead of S, −16 amu) in the FLP peak was measured with HRMS.

| Compound | | | | | | % -16 |
|---|---|---|---|---|---|---|
| (SEQ ID | | Calculated | Found | OD / | | amu in |
| NO) | Compound sequence | mass | mass | μmol | % FLP | FLP |
| Control | TCAGtttcactattcGTACT | 6920.6 | 6919.7 | 126 | 84.4 | 3.2 |
| (12) | | | | | | |
| Seq-1 (1) | T_{PS2}CAGtttcactattcGTACT | 6936.7 | 6935.8 | 129 | 48.2 | 4 |
| Seq-2 (2) | TC_{PS2}AGtttcactattcGTACT | 6936.7 | 6935.8 | 130 | 50.8 | 7.9 |
| Seq-3 (3) | TCA_{PS2}GtttcactattcGTACT | 6936.7 | 6935.8 | 133 | 57.5 | 6.7 |
| Seq-4 (4) | TCAG_{PS2}tttcactattcGTACT | 6936.7 | 6935.8 | 129 | 64 | 4.9 |
| Seq-5 (5) | TCAGtttcactattcGTAC_{PS2}T | 6936.7 | 6935.8 | 136 | 85.8 | 4.9 |
| Seq-6 (6) | TCAGtttcactattcGT_{PS2}CT | 6936.7 | 6935.8 | 131 | 87.4 | 5.1 |
| Seq-7 (7) | TCAGtttcactattcGT_{PS2}ACT | 6936.7 | 6935.8 | 127 | 78.6 | 4.4 |
| Seq-8 (8) | TCAGtttcactattcG_{PS2}TACT | 6936.7 | 6935.8 | 130 | 88.1 | 4.7 |
| Seq-9 (9) | T_{PS2}CAGtttcactattcGTAC_{PS2}T | 6952.8 | 6951.9 | 130 | 49.7 | 6.7 |
| Seq-10 (10) | TpoC_{PO}AGtttcactattc_{PS2}TACT | 6904.6 | 6902.9 | 118 | 86.0 | 5.5 |
| Seq-11 (11) | T_{PO}C_{PO}A_{PO}G_{PO}tttcactattc_{PS2}TACT | 6872.5 | 6872.1 | 120 | 85.0 | 6.2 |

_{PS2} denotes phosphorodithioate and _{PO} denotes phosphate linkage between adjacent nucleosides, all other inter nucleosidic linkages were phosphorothioates
A, T, G, ^{m}c denotes LNA nucleosides
a, t, g, c denotes DNA nucleosides

1. (canceled)

2. (canceled)

3. (canceled)

4. (canceled)

5. A method for the preparation of an oligonucleotide containing at least one phosphorodithioate internucleoside linkage, said method comprising the steps of:
coupling a compound of formula (I): 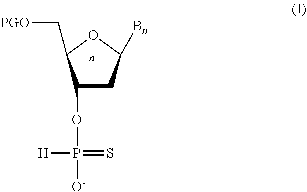
wherein—in formula (I)—PG is a hydroxyl-protecting group, Bn is a nucleobase that can be natural or non-natural, and wherein the ribose n is modified at the 2′ position of its ring,
with a compound of formula (II): 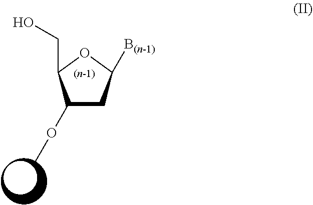
wherein—in formula (II)—B(n−1) is a nucleobase that can be natural or non-natural, and
wherein ribose (n−1) can be unmodified or modified at the 2′ position of its ring,
in the presence of a coupling agent, to obtain a compound of formula (III):

6. The method according to claim 5 further comprising the steps of: 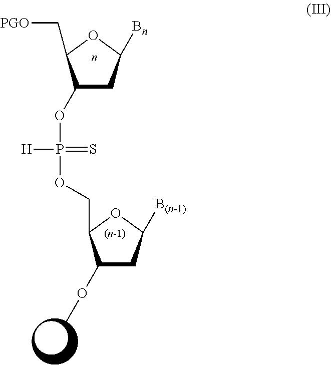
a) performing a thioalkylation of the compound of formula (III) at the hydrogen bound to the phosphorus atom, to obtain a compound of formula (IVa): 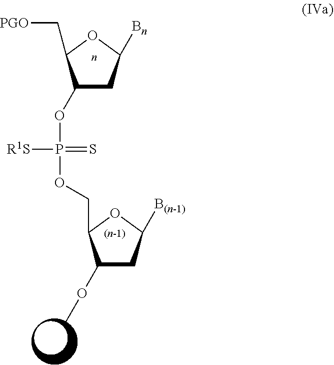
wherein R¹ is a cyanoalkyl, and wherein PG, Bn, B(n−1) and the riboses n and (n−1) are as defined for Formula (III);
b) or performing a reaction of the compound of formula (III) with a xanthane hydride to obtain a compound of formula (IVb): 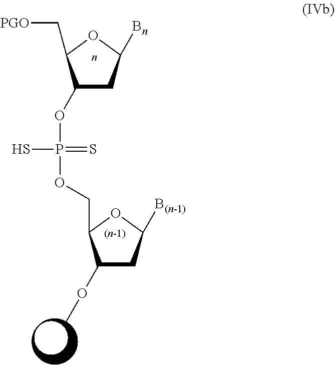
wherein PG, Bn, B(n−1) and the riboses n and (n−1) are as defined for Formula (III).

7. The method according to claim 6, further comprising the step of:
cleaving and deprotecting the compound of formula (IVa) or (IVb) to obtain a compound of formula (V): 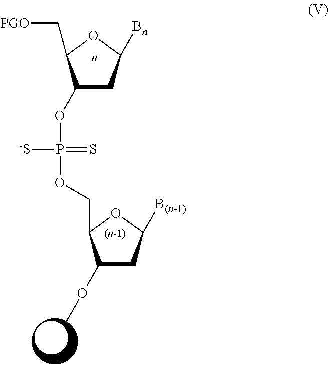
wherein PG, Bn, B(n−1) and the riboses n and (n−1) are as defined for Formula (III).

8. The method according to claim 6, further comprising the step of:
deprotecting the compound of formula (IVa) to provide a compound of formula (VIa), 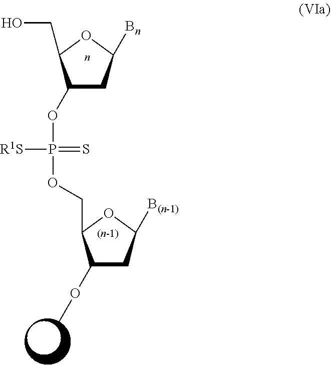
wherein Bn, B(n−1) and the riboses n and (n−1) are as defined for Formula (III);
followed by coupling a compound of formula (I) as defined above, or a 5′-O-protected ribonucleoside phosphoramidite, to the deprotected C5′-OH of the compound of formula (VIa) in the presence of a coupling agent; or deprotecting the compound of formula (IVb) to provide a compound of formula (VIb), 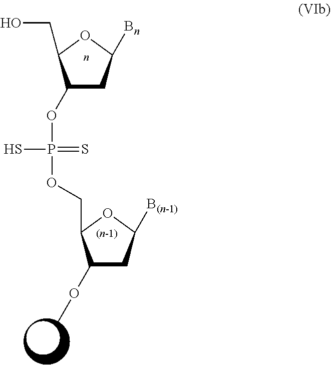
wherein Bn, B(n−1) and the riboses n and (n−1) are as defined for Formula (III);
followed by coupling a compound of formula (I) as defined above, or a 5′-O-protected ribonucleoside phosphoramidite, to the deprotected C5′-OH of the compound of formula (VIb) in the presence of a coupling agent; or,deprotecting the compound of formula (III), to provide a compound of formula 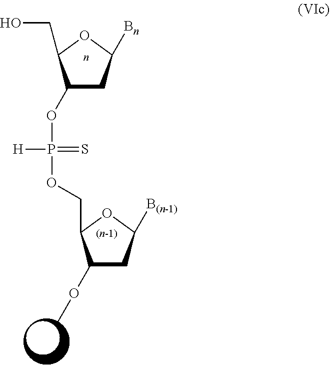
wherein Bn, B(n−1) and the riboses n and (n−1) are as defined for Formula (III); followed by coupling a compound of formula (I) as defined above, or a 5′-O-protected ribonucleoside phosphoramidite, to the deprotected C5′-OH of the compound of formula (VIc) in the presence of a coupling agent.

9. The method according to claim 5, further comprising the step of:
performing a global sulfurization of the compound of formula (III), followed by steps of cleaving and deprotecting to obtain a compound of formula (V):

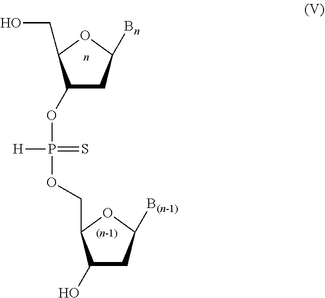

10. The method of claim 5, wherein the coupling agent is a dialkylchlorophosphate, preferably diethyl chlorophosphate, in combination with pyridine.

11. A phosphonothioate monoester selected from the group consisting of: 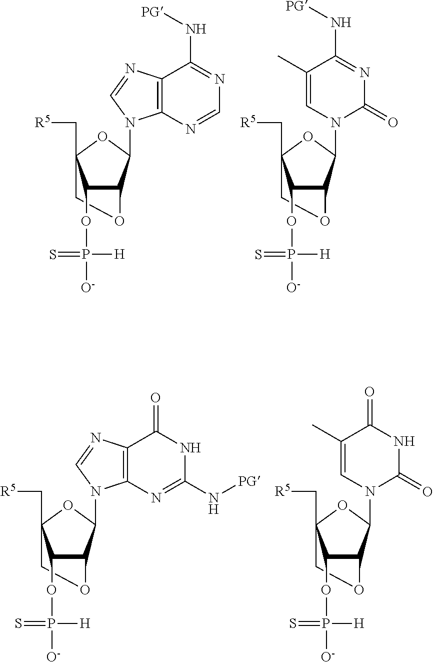
wherein PG′ is an amino-protecting group and R⁵ is H or a hydroxyl-protecting group.

12. The phosphonothioate monoester of claim 11, wherein the phosphonothioate monoester is a phosphonothioate monoester salt selected from the group consisting of: 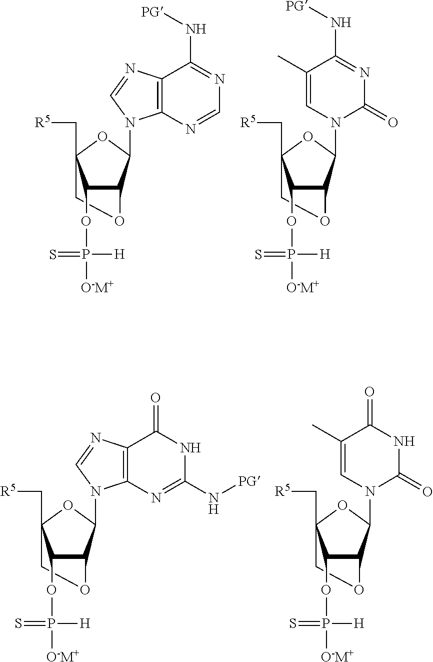
wherein PG′ is an amino-protecting group, M⁺ is a monovalent cation and R⁵ is H or a hydroxyl-protecting group.

13. The method according to claim 5, wherein the modification at the 2′ position of ribose n is selected from the group consisting of locked nucleic acid (LNA), constrained ethyl (cET), 2′-O-methoxyethyl (2′-O-MOE), 2′-O-Methyl and 2′-fluoro modifications, preferably LNA modification.

14. The method according to claim 5 for the preparation of oligonucleotide comprising at least one phosphorodithioate and at least one phosphorothioate internucleoside linkage.

15. The method according to claim 5 for the preparation of an oligonucleotide comprising at least one phosphorodithioate, at least one phosphorothioate and/or at least one phosphodiester internucleoside linkage.


SEQUENCE LISTING (12 sequences)

SEQ ID NO: 1 - DNA, 20 bp, synthetic construct
  misc_feature 1..20: note = Synthetic
  modified_base 1..4: mod_base = OTHER; note = LNA nucleoside
  modified_base 1..2: mod_base = OTHER; note = PS2 - Phosphorodithioate linkage
  modified_base 2..20: mod_base = OTHER; note = Phosphorothioate linkage
  modified_base 16..20: mod_base = OTHER; note = LNA nucleoside
  source 1..20: mol_type = other DNA
tcagtttcactattcgtact

SEQ ID NO: 2 - DNA, 20 bp, synthetic construct
  misc_feature 1..20: note = Synthetic
  modified_base 1..4: mod_base = OTHER; note = LNA nucleoside
  modified_base 1..2: mod_base = OTHER; note = Phosphorothioate linkage
  modified_base 2..3: mod_base = OTHER; note = PS2 - Phosphorodithioate linkage
  modified_base 3..20: mod_base = OTHER; note = Phosphorothioate linkage
  modified_base 16..20: mod_base = OTHER; note = LNA nucleoside
  source 1..20: mol_type = other DNA
tcagtttcactattcgtact

SEQ ID NO: 3 - DNA, 20 bp, synthetic construct
  misc_feature 1..20: note = Synthetic
  modified_base 1..4: mod_base = OTHER; note = LNA nucleoside
  modified_base 1..3: mod_base = OTHER; note = Phosphorothioate linkage
  modified_base 3..4: mod_base = OTHER; note = PS2 - Phosphorodithioate linkage
  modified_base 4..20: mod_base = OTHER; note = Phosphorothioate linkage
  modified_base 16..20: mod_base = OTHER; note = LNA nucleoside
  source 1..20: mol_type = other DNA
tcagtttcactattcgtact

SEQ ID NO: 4 - DNA, 20 bp, synthetic construct
  misc_feature 1..20: note = Synthetic
  modified_base 1..4: mod_base = OTHER; note = LNA nucleoside
  modified_base 1..4: mod_base = OTHER; note = Phosphorothioate linkage
  modified_base 4..5: mod_base = OTHER; note = PS2 - Phosphorodithioate linkage
  modified_base 5..20: mod_base = OTHER; note = Phosphorothioate linkage
  modified_base 16..20: mod_base = OTHER; note = LNA nucleoside
  source 1..20: mol_type = other DNA
tcagtttcactattcgtact

SEQ ID NO: 5 - DNA, 20 bp, synthetic construct
  misc_feature 1..20: note = Synthetic
  modified_base 1..4: mod_base = OTHER; note = LNA nucleoside
  modified_base 1..19: mod_base = OTHER; note = Phosphorothioate linkage
  modified_base 16..20: mod_base = OTHER; note = LNA nucleoside
  modified_base 19..20: mod_base = OTHER; note = P PS2 - hosphorodithioate linkage
  source 1..20: mol_type = other DNA
tcagtttcactattcgtact

SEQ ID NO: 6 - DNA, 19 bp, synthetic construct
  misc_feature 1..19: note = Synthetic
  modified_base 1..4: mod_base = OTHER; note = LNA nucleoside
  modified_base 1..17: mod_base = OTHER; note = Phosphorothioate linkage
  modified_base 16..19: mod_base = OTHER; note = LNA nucleoside
  modified_base 17..18: mod_base = OTHER; note = PS2 - Phosphorodithioate linkage
  modified_base 18..19: mod_base = OTHER; note = Phosphorothioate linkage
  source 1..19: mol_type = other DNA
tcagtttcactattcgtct

SEQ ID NO: 7 - DNA, 20 bp, synthetic construct
  misc_feature 1..20: note = Synthetic
  modified_base 1..4: mod_base = OTHER; note = LNA nucleoside
  modified_base 1..17: mod_base = OTHER; note = Phosphorothioate linkage
  modified_base 16..20: mod_base = OTHER; note = LNA nucleoside
  modified_base 17..18: mod_base = OTHER; note = PS2 - Phosphorodithioate linkage
  modified_base 18..20: mod_base = OTHER; note = Phosphorothioate linkage
  source 1..20: mol_type = other DNA
tcagtttcactattcgtact

SEQ ID NO: 8 - DNA, 20 bp, synthetic construct
  misc_feature 1..20: note = Synthetic
  modified_base 1..4: mod_base = OTHER; note = LNA nucleoside
  modified_base 1..16: mod_base = OTHER; note = Phosphorothioate linkage
  modified_base 16..20: mod_base = OTHER; note = LNA nucleoside
  modified_base 16..17: mod_base = OTHER; note = PS2 - Phosphorodithioate linkage
  modified_base 17..20: mod_base = OTHER; note = Phosphorothioate linkage
  source 1..20: mol_type = other DNA
tcagtttcactattcgtact

SEQ ID NO: 9 - DNA, 20 bp, synthetic construct
  misc_feature 1..20: note = Synthetic
  modified_base 1..4: mod_base = OTHER; note = LNA nucleoside
  modified_base 1..2: mod_base = OTHER; note = PS2 - Phosphorodithioate linkage
  modified_base 2..19: mod_base = OTHER; note = Phosphorothioate linkage
  modified_base 16..20: mod_base = OTHER; note = LNA nucleoside
  modified_base 19..20: mod_base = OTHER; note = PS2 - Phosphorodithioate linkage
  source 1..20: mol_type = other DNA
tcagtttcactattcgtact

SEQ ID NO: 10 - DNA, 19 bp, synthetic construct
  misc_feature 1..19: note = Synthetic
  modified_base 1..4: mod_base = OTHER; note = LNA nucleoside
  modified_base 3..15: mod_base = OTHER; note = Phosphorothioate linkage
  modified_base 15..16: mod_base = OTHER; note = PS2 - Phosphorodithioate linkage
  modified_base 16..19: mod_base = OTHER; note = LNA nucleoside
  modified_base 16..19: mod_base = OTHER; note = Phosphorothioate linkage
  source 1..19: mol_type = other DNA
tcagtttcactattctact

SEQ ID NO: 11 - DNA, 19 bp, synthetic construct
  misc_feature 1..19: note = Synthetic
  modified_base 1..4: mod_base = OTHER; note = LNA nucleoside
  modified_base 5..15: mod_base = OTHER; note = Phosphorothioate linkage
  modified_base 15..16: mod_base = OTHER; note = PS2 - Phosphorodithioate linkage
  modified_base 16..19: mod_base = OTHER; note = LNA nucleoside
  modified_base 16..19: mod_base = OTHER; note = Phosphorothioate linkage
  source 1..19: mol_type = other DNA
tcagtttcactattctact

SEQ ID NO: 12 - DNA, 20 bp, synthetic construct
  misc_feature 1..20: note = Synthetic
  modified_base 1..4: mod_base = OTHER; note = LNA nucleoside
  modified_base 1..20: mod_base = OTHER; note = Phosphorothioate linkages
  modified_base 16..20: mod_base = OTHER; note = LNA nucleoside
  source 1..20: mol_type = other DNA
tcagtttcactattcgtact